SEQUENCE LISTING (73 sequences)

SEQ ID NO: 1 - DNA, 19 bp, Artificial Sequence
  Description of Artificial Sequence Synthetic primer
agtgaagacacggccgggt

SEQ ID NO: 2 - DNA, 20 bp, Artificial Sequence
  Description of Combined DNA/RNA Molecule Synthetic primer
atggtttgugtgaactcttc

SEQ ID NO: 3 - DNA, 20 bp, Artificial Sequence
  Description of Combined DNA/RNA Molecule Synthetic primer
acaaaccautgcgttatggt

SEQ ID NO: 4 - DNA, 19 bp, Artificial Sequence
  Description of Combined DNA/RNA Molecule Synthetic primer
accgtgcguatattaaatc

SEQ ID NO: 5 - DNA, 19 bp, Artificial Sequence
  Description of Combined DNA/RNA Molecule Synthetic primer
acgcacgguacaactaagc

SEQ ID NO: 6 - DNA, 21 bp, Artificial Sequence
  Description of Combined DNA/RNA Molecule Synthetic primer
atgtcttucaacgagtacctc

SEQ ID NO: 7 - DNA, 17 bp, Artificial Sequence
  Description of Combined DNA/RNA Molecule Synthetic primer
aaagacaugggcttgta

SEQ ID NO: 8 - DNA, 20 bp, Artificial Sequence
  Description of Combined DNA/RNA Molecule Synthetic primer
actcggatutatcctttggc

SEQ ID NO: 9 - DNA, 17 bp, Artificial Sequence
  Description of Combined DNA/RNA Molecule Synthetic primer
aatccgagugagtgcca

SEQ ID NO: 10 - DNA, 18 bp, Artificial Sequence
  Description of Combined DNA/RNA Molecule Synthetic primer
atattcgucacactgcac

SEQ ID NO: 11 - DNA, 19 bp, Artificial Sequence
  Description of Combined DNA/RNA Molecule Synthetic primer
acgaatauagcgaacaact

SEQ ID NO: 12 - DNA, 18 bp, Artificial Sequence
  Description of Combined DNA/RNA Molecule Synthetic primer
attcctctgucttccaat

SEQ ID NO: 13 - DNA, 18 bp, Artificial Sequence
  Description of Combined DNA/RNA Molecule Synthetic primer
acagaggaaucttggttc

SEQ ID NO: 14 - DNA, 17 bp, Artificial Sequence
  Description of Combined DNA/RNA Molecule Synthetic primer
acgagccugtagtatac

SEQ ID NO: 15 - DNA, 17 bp, Artificial Sequence
  Description of Artificial Sequence Synthetic primer
tgaagacacggccgggt

SEQ ID NO: 16 - DNA, 45 bp, Artificial Sequence
  Description of Artificial Sequence Synthetic primer
gaggtcaccgccatcgcagatcgctagcaatatcaggagattttg

SEQ ID NO: 17 - DNA, 45 bp, Artificial Sequence
  Description of Artificial Sequence Synthetic primer
caaaatctcctgatattgctagcgatctgcgatggcggtgacctc

SEQ ID NO: 18 - DNA, 27 bp, Artificial Sequence
  Description of Artificial Sequence Synthetic primer
gaggtcaccgccatcgcagatcgctag

SEQ ID NO: 19 - DNA, 27 bp, Artificial Sequence
  Description of Artificial Sequence Synthetic primer
ctagcgatctgcgatggcggtgacctc

SEQ ID NO: 20 - DNA, 27 bp, Artificial Sequence
  Description of Artificial Sequence Synthetic primer
ctagcgatctgcgatngcggtgacctc

SEQ ID NO: 21 - DNA, 34 bp, Artificial Sequence
  Description of Artificial Sequence Synthetic primer
catcgcagatcgctancaatatcaggagattttg

SEQ ID NO: 22 - DNA, 34 bp, Artificial Sequence
  Description of Artificial Sequence Synthetic primer
cttcgcagatcgctagcaatatcaggagattttg

SEQ ID NO: 23 - DNA, 34 bp, Artificial Sequence
  Description of Artificial Sequence Synthetic primer
caaaatctcctgatattgctagcgatctgcgatg

SEQ ID NO: 24 - DNA, 11 bp, Artificial Sequence
  Description of Artificial Sequence Synthetic primer
gcggtgacctc

SEQ ID NO: 25 - DNA, 18 bp, Artificial Sequence
  Description of Artificial Sequence Synthetic primer
caatatcaggagattttg

SEQ ID NO: 26 - DNA, 54 bp, Artificial Sequence
  Description of Combined DNA/RNA Molecule Synthetic primer
nnnatcgcagatcgctagcaatnnnnnnnnnnnnnnaagacacggccgggtnnn

SEQ ID NO: 27 - DNA, 54 bp, Artificial Sequence
  Description of Combined DNA/RNA Molecule Synthetic primer
nnnacccggccgtgtcttnnnnnnnnnnnnnnattgctagcgatctgcgatnnn

SEQ ID NO: 28 - DNA, 22 bp, Artificial Sequence
  Description of Combined DNA/RNA Molecule Synthetic primer
nnnatcgcagatcgctagcaat

SEQ ID NO: 29 - DNA, 22 bp, Artificial Sequence
  Description of Artificial Sequence Synthetic primer
taagctagcgatctgcgatnnn

SEQ ID NO: 30 - DNA, 18 bp, Artificial Sequence
  Description of Artificial Sequence Synthetic primer
ctagcgatctgcnatnnn

SEQ ID NO: 31 - DNA, 17 bp, Artificial Sequence
  Description of Artificial Sequence Synthetic primer
cgcagatcgctancaat

SEQ ID NO: 32 - DNA, 48 bp, Artificial Sequence
  Description of Combined DNA/RNA Molecule Synthetic primer
atcgcagatcgctagcaatnnnnnnnnnnnnnnaagacacggccgggt

SEQ ID NO: 33 - DNA, 48 bp, Artificial Sequence
  Description of Combined DNA/RNA Molecule Synthetic primer
acccggccgtgtcttnnnnnnnnnnnnnnattgctagcgatctgcgat

SEQ ID NO: 34 - DNA, 14 bp, Artificial Sequence
  Description of Artificial Sequence Synthetic primer
cccggccgtntctt

SEQ ID NO: 35 - DNA, 14 bp, Artificial Sequence
  Description of Artificial Sequence Synthetic primer
cacggccggntnnn

SEQ ID NO: 36 - DNA, 18 bp, Artificial Sequence
  Description of Combined DNA/RNA Molecule Synthetic primer
aagacacggccgggtnnn

SEQ ID NO: 37 - DNA, 18 bp, Artificial Sequence
  Description of Combined DNA/RNA Molecule Synthetic primer
nnnacccggccgtgtctt

SEQ ID NO: 38 - DNA, 16 bp, Artificial Sequence
  Description of Combined DNA/RNA Molecule Synthetic primer
nnncgcagatcgctag

SEQ ID NO: 39 - DNA, 16 bp, Artificial Sequence
  Description of Artificial Sequence Synthetic primer
ctagcgatctgcnnnn

SEQ ID NO: 40 - DNA, 45 bp, Artificial Sequence
  Description of Artificial Sequence Synthetic primer
cgcagatcgctancaatnnnnnnnnnnnnnnaagacacggccggg

SEQ ID NO: 41 - DNA, 45 bp, Artificial Sequence
  Description of Artificial Sequence Synthetic primer
cccggccgtntcttnnnnnnnnnnnnnnattgctagcgatctgcg

SEQ ID NO: 42 - DNA, 14 bp, Artificial Sequence
  Description of Combined DNA/RNA Molecule Synthetic primer
nnnacccggccgtg

SEQ ID NO: 43 - DNA, 18 bp, Artificial Sequence
  Description of Combined DNA/RNA Molecule Synthetic primer
nnnatcgcagatcgctag

SEQ ID NO: 44 - DNA, 32 bp, Artificial Sequence
  Description of Combined DNA/RNA Molecule Synthetic primer
caatnnnnnnnnnnnnnnaagacacggccggg

SEQ ID NO: 45 - DNA, 35 bp, Artificial Sequence
  Description of Combined DNA/RNA Molecule Synthetic primer
tcttnnnnnnnnnnnnnnattgctagcgatctgcg

SEQ ID NO: 46 - DNA, 31 bp, Artificial Sequence
  Description of Artificial Sequence Synthetic primer
gaggtcaccgccatcgcagatcgctagcaat

SEQ ID NO: 47 - DNA, 31 bp, Artificial Sequence
  Description of Artificial Sequence Synthetic primer
attgctagcgatctgcgatggcggtgacctc

SEQ ID NO: 48 - DNA, 31 bp, Artificial Sequence
  Description of Combined DNA/RNA Molecule Synthetic primer
attgctagcgatctgcgauggcggtgacctc

SEQ ID NO: 49 - DNA, 33 bp, Artificial Sequence
  Description of Combined DNA/RNA Molecule Synthetic primer
atcgcagatcgctagcaauatcaggagattttg

SEQ ID NO: 50 - DNA, 33 bp, Artificial Sequence
  Description of Artificial Sequence Synthetic primer
atcgcagatcgctagcaatatcaggagattttg

SEQ ID NO: 51 - DNA, 33 bp, Artificial Sequence
  Description of Artificial Sequence Synthetic primer
caaaatctcctgatattgctagcgatctgcgat

SEQ ID NO: 52 - DNA, 12 bp, Artificial Sequence
  Description of Artificial Sequence Synthetic primer
ggcggtgacctc

SEQ ID NO: 53 - DNA, 14 bp, Artificial Sequence
  Description of Artificial Sequence Synthetic primer
atcaggagattttg

SEQ ID NO: 54 - DNA, 19 bp, Artificial Sequence
  Description of Combined DNA/RNA Molecule Synthetic primer
taagctagcgatctgcgau

SEQ ID NO: 55 - DNA, 19 bp, Artificial Sequence
  Description of Combined DNA/RNA Molecule Synthetic primer
atcgcagatcgctagcaau

SEQ ID NO: 56 - DNA, 15 bp, Artificial Sequence
  Description of Combined DNA/RNA Molecule Synthetic primer
acccggccgtgtctu

SEQ ID NO: 57 - DNA, 15 bp, Artificial Sequence
  Description of Combined DNA/RNA Molecule Synthetic primer
aagacacggccgggu

SEQ ID NO: 58 - DNA, 22 bp, Artificial Sequence
  Description of Combined DNA/RNA Molecule Synthetic primer
attgctagcgatctgcgaunnn

SEQ ID NO: 59 - DNA, 48 bp, Artificial Sequence
  Description of Combined DNA/RNA Molecule Synthetic primer
atcgcagatcgctagcaaunnnnnnnnnnnnnnaagacacggccgggt

SEQ ID NO: 60 - DNA, 48 bp, Artificial Sequence
  Description of Combined DNA/RNA Molecule Synthetic primer
acccggccgtgtctunnnnnnnnnnnnnnattgctagcgatctgcgat

SEQ ID NO: 61 - DNA, 18 bp, Artificial Sequence
  Description of Combined DNA/RNA Molecule Synthetic primer
aagacacggccgggunnn

SEQ ID NO: 62 - DNA, 29 bp, Artificial Sequence
  Description of Artificial Sequence Synthetic primer
nnnnnnnnnnnnnnaagacacggccgggt

SEQ ID NO: 63 - DNA, 33 bp, Artificial Sequence
  Description of Artificial Sequence Synthetic primer
nnnnnnnnnnnnnnattgctagcgatctgcgat

SEQ ID NO: 64 - DNA, 32 bp, Artificial Sequence
  Description of Combined DNA/RNA Molecule Synthetic primer
ggagacauaaatcttttgctctctcttcctgc

SEQ ID NO: 65 - DNA, 33 bp, Artificial Sequence
  Description of Combined DNA/RNA Molecule Synthetic primer
gatattgctagcgauctgcgatggcggtgacct

SEQ ID NO: 66 - DNA, 34 bp, Artificial Sequence
  Description of Combined DNA/RNA Molecule Synthetic primer
atcgctagcaataucaggagattttgattttttg

SEQ ID NO: 67 - DNA, 33 bp, Artificial Sequence
  Description of Combined DNA/RNA Molecule Synthetic primer
ctcacccggccgtgucttcactaaactcctagc

SEQ ID NO: 68 - DNA, 33 bp, Artificial Sequence
  Description of Combined DNA/RNA Molecule Synthetic primer
aagacacggccgggugagaattggttttctttc

SEQ ID NO: 69 - DNA, 32 bp, Artificial Sequence
  Description of Combined DNA/RNA Molecule Synthetic primer
gggaaaguttaatttcttgaaattttccagat

SEQ ID NO: 70 - DNA, 18 bp, Artificial Sequence
  Description of Combined DNA/RNA Molecule Synthetic primer
cuagcgaucugcgatnnn

SEQ ID NO: 71 - DNA, 17 bp, Artificial Sequence
  Description of Combined DNA/RNA Molecule Synthetic primer
cgcagaucgcuagcaat

SEQ ID NO: 72 - DNA, 14 bp, Artificial Sequence
  Description of Combined DNA/RNA Molecule Synthetic primer
cccggccgugtctt

SEQ ID NO: 73 - DNA, 45 bp, Artificial Sequence
  Description of Combined DNA/RNA Molecule Synthetic primer
cccggccgugtcttnnnnnnnnnnnnnnattgctagcgatctgcg

(19) United States
(12) Patent Application Publication
(10) Pub. No.: US 2008/0213841 A1
(43) Pub. Date: Sep. 4, 2008

(54) Novel Method for Assembling DNA Metasegments to use as Substrates for Homologous Recombination in a Cell

(75) Inventor: Srinivasan Chandrasegaran, Towson, MD (US)

Correspondence Address: CHERYL H AGRIS PHD, PO BOX 806, PELHAM, NY, 10803, US

(73) Assignee: PONDICHERRY BIOTECH PRIVATE LIMITED, Pondicherry (IN)

(21) Appl. No.: 11/828,970

(22) Filed: Jul. 26, 2007

Related U.S. Application Data
(60) Provisional application No. 60/839,101, filed on Aug. 21, 2006.

(51) Int. Cl. C12P 19/34; C12N 9/10

(52) U.S. Cl. 435/91.1; 435/193

(57) ABSTRACT

The invention relates to a novel method for obtaining DNA metasegment comprising ligating adjoining DNA fragments at least 10 Kb in size containing at least one overlapping region.

### PRIORITY CLAIM

This application claims priority to provisional application No. 60/839,101, filed Aug. 21, 2006, the contents of which are incorporated herein by reference.

### FIELD OF THE INVENTION

The invention is directed to a novel DNA metasegment assembly process wherein multiple overlapping adjoining DNA fragments are assembled into a DNA metasegments. Such DNA metasegment products generated may be used as a substrate for homologous recombination in a cell.

### BACKGROUND OF THE INVENTION

Recent advances in synthetic biology have made it possible to design and synthesize DNA fragments of about 10 Kb or more in size (Endy, 2005; Chin, 2006; Posfai et al 2006). This not only makes it feasible to design and chemically synthesize large plant and mammalian genes with the desired alterations (Menzella et al 2005); but also makes it possible to assemble large DNA metasegments to use as donor DNA for homologous recombination in a cell for targeted re-engineering of the genome of a cell and to use them as building blocks to create a synthetic chromosome and eventually a synthetic genome of a cell. Synthetic oligodeoxyribonucleotides have been used to generate the whole genome of φX174 bacteriophage (Smith et al 2003).

However, an efficient and effective method to assemble large DNA metasegments from smaller 0.5 to 10-Kb synthetic DNA fragments is required to create a synthetic chromosome and eventually a synthetic genome of a cell.

A method has recently been reported for sequential iterative recombination approach for replacing wild type sequences of a genome in a cell with synthetic DNA metasegments at least in organisms like yeast that show a high frequency of homologous recombination (http://macbeth.clark.jhu.edu/syntheticyeast/wiki/index.php/Sc2.0). In this case, the synthetic DNA metasegment was assembled using a classical approach: unique restriction enzyme sites were designed into the synthetic fragments to enable ligation of the smaller DNA fragments. This is not a very efficient process since ligation yield is normally low and other unwanted side-products result from self-ligation of substrates.

### SUMMARY OF THE INVENTION

The invention relates to a novel process for easy assembly of large DNA metasegments from smaller 0.3-10-Kb, or more particularly, 0.5-10 Kb, synthetic or naturally occurring DNA fragments; and novel uses for the assembled DNA metasegments: for use in targeted engineering of the genome of a cell and for potential use as building blocks (“bricks and mortar”) for creating a synthetic chromosome, and eventually a synthetic genome of a cell and novel use for the re-engineered or synthetic chromosomes or genomes created using the novel DNA metasegment assembly process described below in genomic transplantation of cells (Lartigue et al 2007).

This novel method, hereafter will be known as DNA MetaSegment Assembly Process (DMSAP) and the Products (DNA metasegments) generated by DMSAP, will be referred to as DMSAPP. This invention in its current embodiment incorporates several new ideas/concepts into the development of this novel process and composition. The major advantage of this novel process is that DMSAPP, the DNA metasegments generated by DMSAP, can be directly introduced into a cell, without a need for cloning them in E. coli or yeast, since cloning of foreign DNA more often than not is toxic to E. coli and yeast.

The method of the invention comprises the following steps:
(a) providing a plurality of adjoining tandem DNA fragments wherein each fragment is at least 0.3 Kb in length, or more particularly, at least 0.5 Kb in length, wherein each adjoining fragment comprises overlapping regions wherein said overlapping region comprises an overlap of at least three base pairs, or more particularly at least four base pairs, five base pairs, six base pairs, seven base pairs, eight base pairs, nine base pairs or ten base pairs, or alternatively 3-20 base pairs (bp) at the 5′ end and/or 3′ end between said adjoining fragments;
(b) contacting the fragments provided in (a) with at least one forward primer and one reverse primer, wherein each primer (i) is sufficiently complementary to the overlapping regions between said adjoining fragments to hybridize to at least one of said adjoining fragments, and (ii) comprises at least one removable base and (iii) is at least 15 nucleotides in length;
(c) amplifying the tandem DNA fragments of (a) using the primers of (b) to obtain amplified fragments, wherein the forward primer is used to amplify the plus strand of one of said DNA fragments and the reverse primer is used to amplify the minus strand of one of said DNA fragments;
(d) removing said removable bases from the amplified fragments of (c) and cleaving one or more phosphodiester bonds at the sites of said removable bases to produce DNA fragments at least 0.3 Kb in length with compatible single-stranded ends at least three nucleotides in length, or more particularly 4, 5, 6, 7, 8, 9 or 10 nucleotides in length or alternatively 3-20 nucleotides in length or 7-15 nucleotides in length, wherein said compatible ends of said DNA fragments are sufficiently complementary to each other to hybridize to one another;
(e) ligating fragments with compatible single-stranded ends generated in (d) to obtain said DNA metasegments;
(f) optionally repeating steps (a)-(e) at least once and
(g) isolating said DNA metasegments.

In one embodiment, two adjoining fragments are provided. In another embodiment, three fragments may be provided. The method of the invention further comprises providing comprises providing at least two fragments, amplifying and ligating and then sequentially adding one or more fragments. As will be described in further detail below, the method of the present invention is automatable.

As will be discussed in further detail below, the fragment used in the method of the present invention may be chemically synthesized or obtained by other methods known in the art, such as PCR. Alternatively, the DNA fragments may be isolated natural DNA fragments from an organism.

The tandem DNA fragments provided in step (a) in a particular embodiment contain an overlap of at least about 3 bp, or more particularly, at least 4, 5, 6, 7, 8, 9 or 10 bp, or alternatively an overlap of between 3-20 bp or between 7-15 bp at the 5′ end and/or 3′ end between the adjoining fragments. In yet another particular embodiment, the overlapping region of the DNA fragments provided in (a) terminates with C at the 5′ end and may further comprise G at least five nucleotides downstream from said C. Alternatively, the overlapping region of the DNA fragments provided in (a) terminates with A at the 5′ end and optionally further comprises T at least five nucleotides downstream from said A.

The primers used in the method of the present invention comprise at least one removable base in at least one primer provided in step (b) is an acid labile base (e.g., deoxyinosine, N7-methyldeoxyguanine), photolabile base, or substrate for enzymatic removal. In a particular embodiment, the removable base is uracil. In yet another particular embodiment, the removable base may be a ribonucleotide or RNA segment or an abasic site. In one embodiment, the designed forward primers start with the selected C at the 5′ end of the overlap region and the chemically removable base, I, replaces the G, which is located at least five nucleotides downstream of the said C. An additional minimum of 10 bases of the target DNA sequence beyond the overlap region 3′ to the said G is incorporated as a part of the forward primers. The reverse primers for the overlap region are similarly designed and synthesized. In another embodiment, the designed primers start with the selected A at the 5′ end of the overlap region, and the enzymatically removable base, U, replaces T, which is located at least five nucleotides downstream of the said A. An additional minimum of 10 bases of the target DNA sequence beyond the overlap region 3′ to the said T may be incorporated as a part of the forward primers; the reverse primers for the overlap region are similarly designed and synthesized. The said forward and reverse primers designed for the overlap region prime opposite strands of tandem target fragments and they may overlap each other by 7 to 15 base pairs at the 3′ and/or 5′ ends, as the case may be.

In one particular embodiment, the method comprises: (1) Chemical synthesis of a series of 0.5 to 10-Kb adjoining fragments with an overlap of 7-15 bp. (2) PCR amplification of the synthetic 0.5 to 10-Kb DNA segments using the appropriate forward and reverse PCR primers containing one or more strategically placed specific base like a deoxyinosine or a photolabile base (for efficient removal of the base by chemical or photochemical treatment to produce an abasic site) (Kupfer and Leumann, 2007). Alternatively, amplification of the synthetic 0.5 to 10-Kb DNA segments may be accomplished by PCR using appropriate forward and reverse PCR primers containing one or more strategically placed specific base like a uracil (for efficient enzymatic removal using New England Biolabs USER enzymes) (Bitinaite et al. 2007; Geu-Flores et al. 2007; Lasken et al. 1996); or PCR primer containing one or more ribonucleotide(s) (or a ribonucleotide or RNA segment) for efficient enzymatic phosphodiester bond cleavage using an RNase (e.g., RNaseH). (3) Selective removal of the acid labile base deoxyinosine (or photolabile base), followed by strand scission at the abasic site of the resulting PCR-amplified DNA, to generate unique compatible single strand ends between the series of overlapping adjoining DNA fragments by using chemical (or photochemical) treatment. Alternatively, selective enzymatic removal of uracil, followed by cleavage of the phosphodiester bond at the abasic site of the resulting PCR-amplified DNA, by using the uracil-specific excision reagents, (e.g., namely Uracil DNA glycosylase (UDG) and the DNA glycosylase-lyase Endo VIII), to produce unique compatible single-strand ends between adjoining tandem DNA segments. In a third approach, selective removal of the RNA segment is achieved by using RNaseH to produce unique compatible single-strand ends between adjoining tandem DNA segments. (4), the use of T4 DNA ligase or thermostable ligase to ligate the series of adjoining 0.5 to 10-Kb DNA fragments with unique compatible single strand ends to produce a large DNA metasegment.

The invention is further directed to a kit comprising at least one forward and reverse primer at least 15 nucleotides in length, wherein the forward and reverse primers are designed for the overlapping region of a series of tandem fragments that are to be ligated and wherein each primer comprises a removable base (e.g., acid lablile base, photolabile base, substrate for enzymatic removal); a DNA ligase, a DNA polymersase, nucleotide triphosphates and buffers. In a particular embodiment, the kit comprises at least one primer which is an RNA/DNA primer and optionally an RNase (e.g., RNase H) and/or reverse transcriptase or a DNA polymerase with inherent reverse transcriptase activity. In another embodiment, at least one of the primers in the kit may comprise the selected C at the 5′ end of an overlapping region and with an I replacing the G that is at least five nucleotides downstream from the C. In other particular embodiments, the primer comprises N7-methyldeoxyguanosine, deoxyguanosine, or uracil. In a specific embodiment, at least one of the primers (primers used to amplify either the 5′ or 3′ end fragments of the desired DNA metasegment) contains a functional group (e.g., biotin) to attach to a solid support and the primer may optionally further comprise a detectable label.

The DMSAPP (the synthetic DNA metasegments resulting from the DMSAP process), produced could serve as substrates for homologous recombination with the genome of a cell, include targeted re-engineering the genome of a cell, creating a synthetic chromosome of a cell and eventually a synthetic genome of a cell and uses of such engineered chromosomes or genomes using DMSAP in but not limited to transplantation of a chromosome or genome of a bacterial or a yeast or a plant or an animal or a mammalian cell including the human cell or a stem cell.

In more detail, the novel applications and uses for the invention, DMSAP and the resulting DMSAPP include, but are not limited to: (i) replacing parts of a genome with the assembled DNA metasegments for targeted re-engineering the genome of a cell; (ii) sequential iterative replacement of the wild type sequences of a chromosome in a cell with assembled synthetic DNA metasegments by homologous recombination, by alternating between two drug or fluorescent markers to monitor recombination at each step, to finally produce a synthetic chromosome of a cell; (iii) sequential iterative replacement of the wild type sequence of a genome in a cell with synthetic DNA metasegments by homologous recombination, alternating between two drug or fluorescent markers to monitor recombination at each step, to finally produce a synthetic genome of a cell. The cells include but not limited to, a bacterial cell, a yeast cell, a fungal cell, an insect cell, a plant cell, a mammalian cell including an animal cell and a human cell or stem cell.

### BRIEF DESCRIPTION OF THE FIGURES

FIG. 1 shows a representation of various enzymatic and chemical methods for generating DNA segments with unique 3′ and 5′ ends that are compatible with adjoining overlapping DNA fragments. (A), Overlapping DNA segments in this enzymatic method are amplified using primers containing uracil (U) and a thermostable DNA polymerase, preferably one that does not possess 3′ terminal transferase activity. The PCR product is then treated with UDG and Endo VIII enzymes (a mixture of which called “USER” could be purchased from New England Biolabs, Inc., USA) to generate DNA fragments with unique 3′ and 5′ single strand ends that are compatible with adjoining tandem DNA segments. (B), Overlapping DNA segments in this enzymatic method are amplified using RNA-DNA primers and a thermostable DNA polymerase, preferably one that does not possess 3′ terminal transferase activity. The amplification is done in presence of a thermostable reverse transcriptase enzyme for copying through the RNA segment of the primers or done using instead a thermostable DNA polymerase with inherent reverse transcriptase activity could be employed. The PCR product is then treated with RNase H enzyme to generate DNA fragments with unique 3′ and 5′ single strand ends that are compatible with adjoining tandem DNA fragments. (C), Overlapping DNA segments in this method are amplified using primers containing inosine (I) and a thermostable DNA polymerase, preferably one that does not possess 3′ terminal transferase activity. The PCR product is then treated with acid for selective removal of deoxyinosine to form an apurinic site; the phosphodiester bonds at this site is then susceptible to cleavage by piperidine, which upon cleavage will generate DNA fragments with unique 3′ and 5′ single strand ends that are compatible with adjoining or tandem DNA fragments.

FIG. 2 shows a scheme for automating the DNA metasegment assembly process (DMSAP). The 5′-biotinylated DNA segment 1 (with circle at the 5′-end) is bound to streptavidin molecules, which are covalently linked to a solid support (shown by S enclosed in a circle) as described in Invitrogen manual for Dynal kilobaseBINDER kit (Product No. 601.01). The streptavidin molecules are shown by the symbol “(”. After each ligation step of the DNA fragment, the excess reagent is washed away from the desired product, which is covalently attached to the solid support, thereby essentially purifying the desired product away from other reagents and maximizing the yield of the product unlike the solution reaction. After ligation of the final DNA fragment and washing away the excess reagent, the desired DNA metasegment is eluted from the column by dissociating the streptavidin-biotin complex from the solid support as described in Invitrogen manual for Dynal kilobaseBINDER kit (Product No. 601.01).

FIG. 3 shows agarose gels of ligation reaction products in solution of (A) three PCR amplified synthetic 10 Kb overlapping fragments and (B) seven PCR amplified synthetic 10 Kb overlapping fragments.

FIG. 4 shows agarose gels of ligation reaction products from automated DMSAP. (A) PCR amplification of ˜0.5 Kb DNA fragment 1 using 5′-biotinylated forward primer and reverse primer containing uracil, dNTPs and Pfu Turbo Cx. (B) Products from the ligation reaction of 5′-biotinylated PCR amplified DNA fragment 1 with four other PCR amplified ˜0.5 Kb overlapping DNA fragments using corresponding forward and reverse primers containing uracil (see Table 1) and Pfu Turbo Cx, then treated with USER enzymes to produce long unique 3′ and 5′ single strand ends between adjoining fragments. The desired 2.5 Kb product (P) is greatly enriched.

### DETAILED DESCRIPTION OF THE INVENTION

In accordance with the present invention there may be employed conventional molecular biology, techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, Gait ed., 1984, “Oligonucleotide Synthesis”; Hames & Higgins eds., 1985, “Nucleic Acid Hybridization”; Hames & Higgins, eds., 1984, Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

### DMSAP

### DNA Fragments

The DNA fragments used in the method of the present invention may in one embodiment be about 0.5 to 10-Kb in size and are overlapping. Determination of primer designs for the overlapping region of adjoining fragments may be made using bioinformatics tools known in the art (see, for example, (http://macbeth.clark.jhu.edu/syntheticyeast/wiki/index.php/Sc2.0).

In a specific embodiment, the 0.3 to 10-Kb, or more particularly, 0.5-10 Kb fragments required for assembly into a DNA metasegment may be chemically synthesized or obtained from naturally occurring DNA by amplification techniques like PCR. To provide compatible ends to the PCR-amplified 0.3-10 Kb, or more particularly, 0.5 to 10-Kb fragments, there may be an overlap of at least 3 bp, 4 bp, 5 bp, 6 bp, 7 bp, 8 bp, 9 bp or 10 bp or more particularly an overlap of 3 bp-20 bp or even more particularly, 7 bp-15 bp at the 5′ and/or 3′ ends between tandem fragments that are to be assembled.

In a most particular embodiment, the overlapping 7-15 bp segments of the plus strand may terminate with a C at the 5′ end which may be separated by about 5-13 nucleotides from a G nucleotide downstream. Alternatively, the overlapping 7-15 bp segments of the plus strand may terminate with an A at the 5′ end, which is separated by about 5-13 nucleotides from an T nucleotide upstream.

### Primers

The forward and reverse PCR primers for each 0.3 Kb-10 Kb fragment or more particularly, for each 0.5 to 10-Kb fragment, containing one or more acid labile base like deoxyinosine per primer are designed in a specific embodiment to amplify the target DNA using a thermophilic DNA polymerase, preferably one free of 3′ terminal transferase activity. The forward and reverse primers are sufficiently complementary to the overlapping region between these fragments so that they hybridize to this overlapping region, particularly during PCR. A polynucleotide “hybridizes” to another polynucleotide, when a single-stranded form of the polynucleotide can anneal to the other polynucleotide under the appropriate conditions of temperature and solution ionic strength (see Sambrook et al., supra). Hybridization of such sequences may be carried out under stringent conditions. “Stringent conditions” or “stringent hybridization conditions” as defined herein are conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55.degree. C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1.×SSC at 60 to 65° C. Optionally, wash buffers may comprise about 0.1% to about 1% SDS. Duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tm can be approximated from the equation of Meinkoth and Wahl (1984) Anal. Biochem. 138:267-284: Tm=81.5° C.+16.6 (log M)+0.41 (% GC)-0.61 (% formamide)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tm is reduced by about 1° C. for each 1% of mismatching; thus, Tₘ, hybridization, and/or wash conditions can be adjusted to hybridize to sequences of the desired identity. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Cold Spring Harbor, N.Y.).

As noted above, in one embodiment, the overlapping segments of the plus strand of one of the DNA fragments to be amplified may terminate with a C at the 5′ end and may in a particular embodiment be separated by about 5-13 nucleotides from a G nucleotide downstream. Thus, in a particular embodiment the forward and reverse primers are complementary to the overlapping region, except for 3′ guanine, which in the primer sequence is replaced with an acid-labile deoxyinosine.

Furthermore, a N7-methyl deoxyguanine could be used instead of a deoxyinosine. The thermophilic DNA polymerase could incorporate a C opposite the N7-methyl deoxyguanine or deoxyinosine in the template strand. An abasic site could also be directly incorporated in the PCR designs for a T, since the thermophilic DNA polymerase will preferentially incorporate adenine opposite the template stand abasic site, as per the “A” rule for base incorporation by polymerases opposite abasic sites.

As noted above, in yet another embodiment, the overlapping 7-15 bp segments of the plus strand may terminate with an A at the 5′ end, which is separated by about 5-13 nucleotides from a T nucleotide downstream. Thus, the forward and reverse PCR primers for, for example, each 0.3 to 10-Kb, or alternatively 0.5-10 Kb fragment may contain at least one enzymatically removable base like uracil per primer are designed to amplify the target DNA using a thermophilic DNA polymerase. The forward and reverse primers are complementary to the overlapping target sequences between the 0.5 to 10-Kb fragments except for the 3′ thymine, which in the primer sequence is replaced by a uracil (U).

In yet another approach, forward and reverse DNA primers containing one or more ribonucleotides (or a ribonucleotide segment, alternatively referred to as an “RNA fragment” or “RNA segment”) may be designed for a sequence for the overlapping 7-15 bp segments between adjoining series of fragments of 0.3 to 10 Kb or more particularly, 0.5-10 Kb DNA fragments that are to be assembled into a large DNA metasegment. The ribonucleotide(s) is placed at least seven nucleotides away from the 5′ end of the overlap region. Alternatively, a segment of ribonucleotides of at least 7 bp long part of the overlap are placed at the 5′ end of the primers.

### Amplification of DNA Fragments

In a particular embodiment, a series of synthetic or natural 0.5 to 10-Kb DNA fragments may then be amplified by PCR using the designed primers and a DNA polymerase, particularly a thermophilic DNA polymerase. In the presence of dNTPs, the thermophilic DNA polymerase will incorporate adenine opposite the template strand uracil or cytosine opposite the template strand deoxyinosine. After PCR amplification, the ends of the series of adjoining overlapping 0.5 to 10-Kb fragments contain one or more uracil (or an acid labile base deoxyinosine) for selective removal at the terminal 7-15 bp sequences that overlap adjoining fragments. In a particular embodiment, the DNA polymerase is free of 3′-terminal transferase activity. Alternatively, the DNA primers may contain one or more ribonucleotides (or a ribonucleotide segment). In such an instance, the reaction mixture is incubated with thermophilic DNA polymerase, followed by treatment with a reverse transcriptase. Alternatively, the target is amplified using the forward and reverse primers using a thermophilic DNA polymerase with inherent reverse transcriptase activity.

### Phosphodiester Bond Cleavage

The acid labile (or photo labile) bases at the overlapping termini of the resulting PCR-amplified products from the series of 0.5 to 10 Kb fragments may then be removed to form abasic sites, followed by phosphodiester bond cleavage by chemical (or photochemical) treatment, to generate unique single strand 3′ or 5′ extensions in the overlap region between adjoining fragments. The chemical treatment may include the use of very dilute formic acid for selective removal of deoxyinosine base to produce an abasic site, followed by treatment with 10% piperidine for phosphodiester bond cleavage at the abasic site, a protocol akin to the one reported by Maxim-Gilbert for chemical sequencing of DNA (Maxam and Gilbert, 1977; 1980), to generate the unique single strand extensions between adjoining DNA fragments. Formic acid weakens the glycosidic bond by protonating the purine ring nitrogen of deoxyinosine, which is then easily displaced by piperidine.

Alternatively, the uracil residues at the overlapping termini of the resulting PCR-amplified products from the series of 0.5 to 10 Kb adjoining fragments may be excised using a uracil excision reagent, such as UDG, which catalyzes the removal of the uracil base, resulting in an abasic site while leaving an intact phosphodiester backbone. DNA glycosylase-lyase Endo VIII is used to break the phosphodiester backbone at the 3′ and 5′ sides of the abasic site releasing a base free deoxyribose. The uracil excision-phosphodiester cleavage may be performed with a mixture of UDG and Endo VIII. The USER (uracil-specific excision reagent) is a mixture of these two enzymes, can be purchased from New England Biolabs, Inc. The USER friendly cloning kit is specifically designed for cloning purposes only. After the phosphodiester bond breakage, the terminal 7-15 bases long oligonucleotides readily dissociate, leaving the 0.5 to 10-Kb PCR fragments with compatible 7-15 bases long unique single-strand extensions at the 3′ and 5′ ends. The generated single-stand extensions are unique and compatible to adjoining or neighboring 10-Kb fragment partners that are to be ligated.

In another approach, PCR amplified product using the DNA primers containing one or more ribonucleotides (or a ribonucleotide segment) and a DNA thermophilic DNA polymerase, followed by treatment with a thermostable reverse transcriptase (or using a thermophilic DNA polymerase with inherent reverse transcriptase activity), is treated with RNaseH to generate the series of DNA fragments with compatible 7-15 bases long 3′ and 5′ extensions to adjoining fragments.

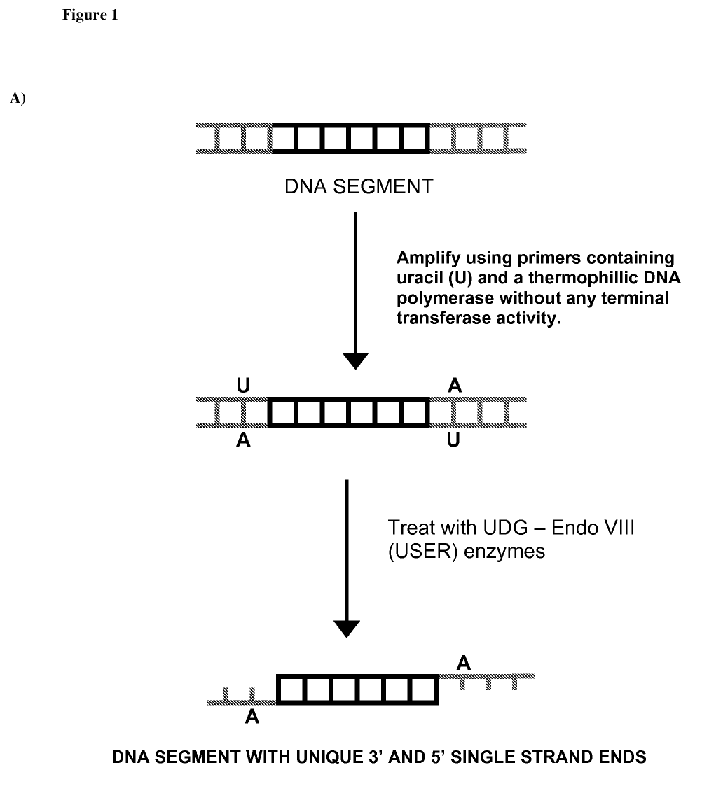

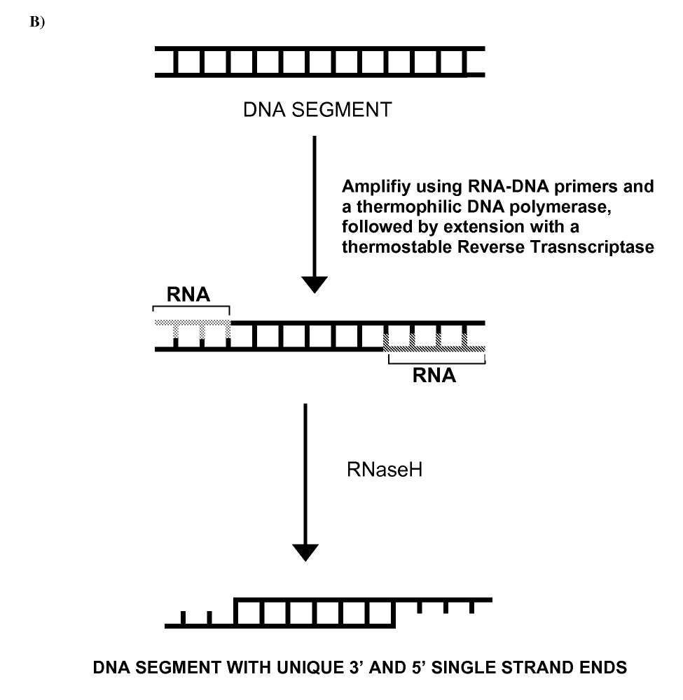

All the three different methods to generate the series of 0.5 to 10 Kb PCR fragments with compatible 7-15 bases long 3′ and 5′ extensions to adjoining fragments are shown schematically in FIG. 1.

### Ligation

The series of amplified DNA fragments which in a specific embodiment are 0.5 to 10-Kb in length with unique 5-15 bases long unique single strand extensions may then pooled together and ligated using a DNA ligase, which includes but is not limited to T4 DNA ligase or a thermostable ligase to produce a DNA metasegment. The PCR amplified 0.5 to 10-Kb fragments with unique long single strand ends could optionally be purified away from the 5-15 bases long oligonucleotides that are released (after the enzymatic UDG-Endo VIII treated 0.5 to 10-Kb PCR DNA fragments with uracil at the termini or chemically treated 0.5 to 10-Kb PCR DNA fragments with deoxyinosine at the termini) and then ligated together. Alternatively, the ligations may be undertaken separately.

In yet another embodiment, the steps described above, providing an adjoining fragment with an overlapping region, amplification, cleavage of phosphodiester bond at the modified base to generate compatible single stranded ends and ligation may be repeated until the metasegment of desired length is obtained.

### Automated DMSAP

The method of the invention may be automated. This automatable method may comprise the following steps:
(a) providing a first DNA fragment at least 0.3 Kb in length;
(b) amplifying said first DNA fragment provided in (a), said amplification using a forward DNA primer containing a functional moiety (eg. Biotin) to enable attachment of the PCR-amplified first 0.3 Kb fragment to a solid support at the 5′ end and a reverse primer, wherein said reverse primer amplifies the minus strand of the said first fragment from the 3′ end, wherein said reverse primer contains one or more removable bases and a DNA polymerase (e.g., thermophilic DNA polymerase without any 3′ terminal transferase activity) to produce a first amplification product;
(c) treating said amplification product of (b) to remove the removable base and cleave the phosphodiester bond at the site of the removable base to generate a first DNA fragment attached to the solid support having a unique 3′ single strand end, wherein said 3′ single strand end is at least 3 nucleotides in length, and may be at least 4, 5, 6, 7, 8, 9 or 10 nucleotides in length or between 3-20 nucleotides in length or more particularly between 7-15 nucleotides in length;
(d) providing a second DNA fragment at least 0.5 Kb in length wherein at least three nucleotides, or more particularly at least 4, 5, 6, 7, 8, 9 or 10 nucleotides or more particularly, between 3-20 nucleotides or 7-15 nucleotides at the 5′ end overlaps said first DNA fragment provided in (a) at the 3′ end;
(e) amplifying said second DNA fragment provided in (d), said amplification using a forward DNA primer and a reverse primer, wherein said forward primer amplifies the plus strand of said second DNA fragment and the reverse primer amplifies the minus strand of said second DNA fragment wherein said primers contain one or more removable bases (e.g., uracils) and a DNA polymerase (e.g., thermophilic DNA polymerase without any 3′ terminal transferase activity) to obtain a second amplified product;
(f) treating said second amplified product of (e) to remove the removable base and cleave a phosphodiester bond at the site of said removable base to generate a second amplified fragment comprising (i) a unique 5′ single stranded end sufficiently complementary to the unique 3′ single stranded end of the first amplified DNA fragment attached to the solid support to hybridize to said 3′ single stranded end and (ii) a unique 3′ single strand end;
(g) ligating the amplified first DNA fragment to the amplified second DNA fragment comprising incubating at least a five fold, and in particular, a 5-10 fold excess of the amplified second DNA fragment with the amplified first DNA fragment in the presence of ligase to produce a first ligation product comprising a 3′ single strand end and then optionally washing away excess reagent;
(h) providing a third DNA fragment at least 0.3 Kb in length, wherein at least three nucleotides, or more particularly, at least four, five, six, seven, eight, nine or ten nucleotides or more particularly, between 3-20 nucleotides or 7-15 at the 5′ end of said third DNA fragment overlaps with the 3′ single strand end of the ligation product of (g);
(i) amplifying said third DNA fragment provided in (h), said amplification using a forward DNA primer and a reverse primer, wherein said forward primer amplifies the plus strand of said third DNA fragment and said reverse primer amplifies the reverse strand of said third DNA fragment, wherein said primers contain one or more removable bases (e.g., uracils and a thermophilic DNA polymerase without any 3′ terminal transferase activity) to obtain a third amplification product;
(j) treating said third amplification product to remove the removable base and cleave a phosphodiester bond at the site of said removable base to generate a third amplified fragment comprising (i) a unique 5′ single stranded end sufficiently complementary to the unique 3′ single stranded end of the first ligation product of (g) attached to the solid support to hybridize to said 3′ single stranded end and (ii) a unique 3′ single stranded end;
(k) ligating the amplified third DNA fragment to the first ligation product of (g) comprising incubating at least a five fold and in particular, a 5-10 fold excess of the amplified third DNA fragment with the first ligation product in the presence of ligase to produce a second ligation product;
(l) optionally repeating steps (d)-(g); to obtain said DNA metasegment and
(m) removing the DNA metasegment obtained from the solid support.

As noted above, the removable base may be an acid labile base (e.g., inosine, N7-methyl-deoxyguanosine), a photolabile base or base susceptible to enzymatic cleavage (e.g., uracil, ribonucleotide(s)).

The method of the invention may further comprise a washing step after one or more of the ligation steps.

The forward primer may have a functional moiety at the 5′ end to enable their attachment to the solid support after PCR amplification. In one embodiment, as set forth below, the forward primer is a biotinylated primer. This biotinylated primer in one embodiment may be bound to streptavidin molecules covalently linked to a solid support.

The solid support may be capillary tubes, beads, fibers, slides, sheets, pins, microtiter plates, silicon, porous silicon, porous metal oxide, plastic, polycarbonate, polystyrene, cellulose, nitrocellulose, nylon, PVDF, glass, TEFLON, polystyrene divinyl benzene, aluminum, carbon, steel, iron, copper, nickel, silver, and gold.

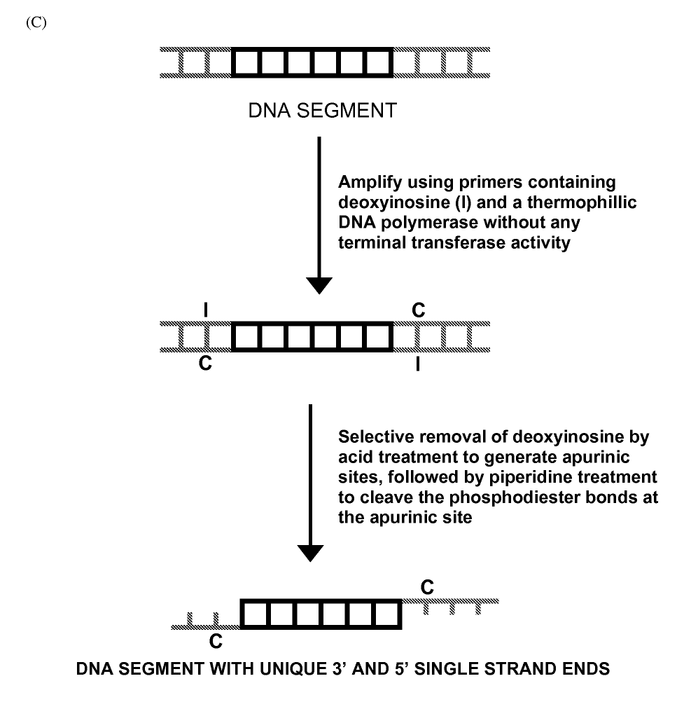

A detailed scheme for automating the DNA metasegment assembly process (DMSAP) is shown in FIG. 2. As a first step, DNA segment is amplified using a 5′-biotinylated forward primer and the 3′ reverse primer contains one or more uracil and is then treated with USER enzymes to generate the unique single stand end as described above. The 5′-biotinylated DNA segment 1 (with circle at the 5′-end) is bound to streptavidin molecules, which are covalently linked to a solid support (for example, using Dynabeads coated with streptavidin which can be purchased from Invitrogen). A 10-20 fold excess of PCR-amplified DNA segment 2 using either DNA primers is added. Where the primer contains uracil, the uracil can be selectively removed by treatment with a uracil excision agent (e.g., USER enzymes namely UDG-Endo VIII). Where the primer comprises one or more ribonucleotides, the ribonucleotides may be selectively removed by RNase treatment (e.g., RNase H). Where the primer comprises a modified nucleotide the modified nucleotide may be selectively removed with chemical treatment. As a result of the removal of these removable bases, a DNA segment 2 is generated with unique 5-15 bases long single strand extensions. DNA segment 2 is then added to the DNA segment 1 attached to solid support in a column and ligated using ATP and T4 DNA ligase (or a thermostable ligase). The excess reagent is then washed away leaving the desired product of ligation of DNA segments 1 and 2. The stepwise addition process is then repeated with PCR amplified and enzymatically or chemically treated DNA segment 3. This cycle is repeated sequentially until the final DNA metasegment is assembled on the solid support. The desired large DNA metasegment is then eluted from the column by dissociating the streptavidin-biotin complex. The other two approaches detailed in FIG. 1 to generate DNA segments with unique compatible 5-15 bases long single strand extensions as reagents for ligation to the DNA fragment 1 attached to the solid support, may also work equally well for automatable DMSAP.

### Uses

Assembled DNA metasegments so generated may serve as valuable substrates for re-engineering of a genome of a cell by homologous recombination. The novel uses for assembled metasegments include but not limited to the following: (i) To replace a wild type sequences of a genome with a synthetic DNA metasegment for targeted re-engineering the genome of a cell; (ii) To replace sequentially in an iterative process, using the DMSAPP encoded with alternating dual selectable drug markers or fluorescent markers, the wild type sequence of a chromosome in a cell with synthetic DNA metasegments, to create a synthetic or artificial chromosome of that cell; (iii) To replace sequentially in an iterative process, using DMSAPP encoded with alternating dual selectable drug markers or fluorescent markers, the wild type DNA sequence of a genome in a cell, to create a synthetic or artificial genome of that cell; (iv) Novel applications for the re-engineered or synthetic chromosomes and genomes using the DMSAP include but limited to transplantation of a chromosome or genome of a prokaryotic or a eukaryotic cell; with a re-engineered or synthetic chromosome or genome; the cells include but not limited to a bacterial cell, or a yeast cell, an insect cell or a plant cell, or a mammalian cell, an animal cell including a human cell and a stem cell.

### EXAMPLES

### Example 1

### Protocol for DNA MetaSegment Assembly Process (DMSAP)

Streptavidin coated solid support is available as Dynabeads Streptavidin from Invitrogen, USA; thermostable reverse transcriptase can be purchased from Epicenter, USA, RNaseH and thermostable ligase from New England Biolabs, USA.

### Experimental Methods and Materials:

The smaller synthetic DNA fragments and the PCR designs containing a single deoxyinosine or a uridine can be custom-ordered and purchased from commercial vendors like Codon Devices, Genescript, etc. or chemically synthesized using a DNA synthesizer.

The smaller synthetic fragments are amplified using the thermostable polymerases like Phusion High-Fidelity DNA polymerase or Phusion Hot Start High-Fidelity DNA polymerase (available at Finnzymes and New England Biolabs) and specifically designed PCR primers. The experimental conditions are as recommended by the manufacturer. Other polymerases that could be used for amplification include, but not limited to, Pfu Turbo Cx polymerase, Deep Vent DNA polymerase or Vent DNA Polymerase (available at New England Biolabs) or GenAmp High Fidelity Enzyme mix (available at Applied Biosystems) or HotStar DNA Taq Polymerase (available at QIAGEN). More specifically, thermostable DNA polymerases that do not add an “A” nucleotide to the 3′ end of the amplified products are highly preferable for use in this methodology.

Selective removal of deoxyinosine base to produce an abasic site, followed by strand scission at the abasic site are achieved by first treating the PCR-amplified product with dilute (5 to 10%) formic acid, and then 10% piperidine treatment at 90° C., to generate the long single strand extensions between adjoining overlapping DNA fragments. This protocol is akin to the one reported by Maxim-Gilbert for chemical sequencing of DNA (Maxam and Gilbert, 1977; 1980).

Selective removal of uracil and strand scission at the abasic site is performed using the USER enzymes that can be procured from New England Biolabs. The experimental conditions are as described in the NEB catalog.

Ligation reactions are performed using T4 DNA ligase (purchased from New England Biolabs) or a thermostable ligase and ATP using conditions for ligation are as recommended by the manufacturer. Ligation reactions using thermostable ligase are done at higher temperatures.

Primers used in the Examples set forth herein are shown in Table 1:

TABLE 1

PRIMERS USED IN PCR AMPLIFICATION OF VARIOUS
~0.5-0.6 Kb DNA FRAGMENTS

| | |
|---|---|
| | DNA |
| | Frag- |
| Forward (For) and Reverse (Rev) Primers | ment # |

| | | |
|---|---|---|
| For 1 | 5′-AGTGAAGACACGGCCGGGT-3′ | 1 |
| (SEQ ID NO: 1) | | |
| Rev 1 | 5′-ATGGTTTGUGTGAACTCTTC-3′ | |
| (SEQ ID NO: 2) | | |
| For 2 | 5′-ACAAACCAUTGCGTTATGGT-3′ | 2 |
| (SEQ ID NO: 3) | | |
| Rev 2 | 5′-ACCGTGCGUATATTAAATC-3′ | |
| (SEQ ID NO: 4): | | |
| For 3 | 5′-ACGCACGGUACAACTAAGC-3′ | 3 |
| (SEQ ID NO: 5): | | |
| Rev 3 | 5′-ATGTCTTUCAACGAGTACCTC-3′ | |
| (SEQ ID NO: 6) | | |
| For 4 | 5′-AAAGACAUGGGCTTGTA-3′ | 4 |
| (SEQ ID NO: 7) | | |
| Rev 4 | 5′-ACTCGGATUTATCCTTTGGC-3′ | |
| (SEQ ID NO: 8) | | |
| For 5 | 5′-AATCCGAGUGAGTGCCA-3′ | 5 |
| (SEQ ID NO: 9) | | |
| Rev 5 | 5′-ATATTCGUCACACTGCAC-3′ | |
| (SEQ ID NO: 10) | | |
| For 6 | 5′-ACGAATAUAGCGAACAACT-3′ | 6 |
| (SEQ ID NO: 11) | | |
| Rev 6 | 5′-ATTCCTCTGUCTTCCAAT-3′ | |
| (SEQ ID NO: 12) | | |
| For 7 | 5′-ACAGAGGAAUCTTGGTTC-3′ | 7 |
| (SEQ ID NO: 13): | | |
| Rev 7 | 5′-ACGAGCCUGTAGTATAC-3′ | |
| (SEQ ID NO: 14) | | |

5′-Biotinylated Forward Primer

| | |
|---|---|
| 5′-Biotin-TGAAGACACGGCCGGGT-3′ | (SEQ ID NO: 15) |

### Step 1: PCR Amplification

Seven 500-600 bp overlapping DNA fragments are amplified using corresponding forward and reverse primers (containing uracil) and Pfu Turbo Cx thermostable DNA polymerase that has no terminal transferase activity (for example Pfu TurboCx). The PCR reaction condition is as follows:

| | |
|---|---|
| Template DNA (10-20 ng) = | 1 μl |
| Forward primer (2 μM) = | 5 μl |
| Reverse primer (2 μM) = | 5 μl |
| 10x buffer = | 10 μl |
| dNTPs (10 mM) = | 2 μl |
| Water = | 65 μl |
| Pfu Turbo Cx = | 2 μl |
| | 100 μl |

The amplification comprises 35 cycles with a denaturing step at 94° C. for 0.5 min, an annealing step at 55° C. for 0.5 min, followed by a polymerase extension step at 72° C. for 2 min. In an initial step before cyclic amplification the DNA is denatured at 94° C. for 2 min and in a final step after cyclic amplification, the polymerase extension is carried out at 72° C. for 10 min. The PCR products were then subjected to QIAtip purification step.

### Step 2: USER Reaction

10 μl of each PCR fragment (containing ˜equimolar of each fragment; ˜0.5-1.0 μg of DNA) are mixed together in an eppendorf tube. 5 μl 3M sodium acetate solution is added and then extracted once, with an equal volume of phenol-chloroform, twice with chloroform and then precipitated with 2.5 volumes of ethanol. The solution is incubated at −80° C. for 1 hr. The resulting precipitate is then spun down using a microcentrifuge at 4° C., washed with 70% ethanol and then air dried for 20 minutes in the hood.

The precipitate is resuspended in 13 μl of water.
Add 2 μl of Taq 10×PCR buffer (from Applied Biosystems, Inc).
1 μl of 100 mM DTT dithiothreitol)
4 μl of USER enzyme (from NEB)
   The mixture is incubated at 37° C. overnight.

Next day, 40 μl of water is added and 5 μl of 3M sodium acetate and conduct phenol-chloroform extraction followed by ethanol precipitation as discussed above.

### Step 3: Annealing Reaction

The USER product from Step 2 is resuspended in 13 μl of water and 4 μl of 5× ligase buffer (from Invitrogen, Inc). The solution is heated at 70° C. for 15-20 minutes, and then the reaction mix is allowed to slowly cool down to room temperature overnight.

### Step 4: Ligation Reaction

Next day, the ligation mix is spun down and then the following mixture is added:
1 μl of 100 mM DTT
1 μl of 10 mM ATP
1 μl of T4 DNA ligase (from NEB)

The ligation mixture is incubated at 16° C. overnight.

### Step 5: Analysis of the DMSAP Products by Agarose Gel Electrophoresis

Next day, the ligation mix is spun down. It is left at room temperature for an hour. Then, 2 μl of the dye is added and the products are analyzed by using a 1% agarose gel electrophoresis.

### Example 2

### Assembly of a 20 Kb Segment from Two Overlapping 10 Kb Fragments with I Containing Primer

In the example described herein, a novel chemical process and composition to assemble two overlapping adjoining 10-Kb fragments to form a 20-Kb segment is described. As further described, the deoxyinosine from the PCR-amplified product is selectively removed by acid treatment to form an abasic site and then the phosphodiester bond at the abasic site is cleaved by chemical treatment using piperidine.

Step 1: Between the adjoining segments, the sequence overlap is chosen such that the plus strands terminate with a G at the 3′ end, which is separated by about 5-13 nucleotides from a C nucleotide upstream (G and C bases are in boldface).

(SEQ ID NO: 16)

5′ . . . GAGGTCACCGCCATCGCAGATCGCTAGCAATATCAGGAGAT
TTTG . . . 3′

(SEQ ID NO: 17)

3′ . . . CTCCAGTGGCGGTAGCGTCTAGCGATCGTTATAGTCCTCTA
AAAC . . . 5′

Step 2: Reverse (R1) and forward (F2) PCR primer designs with a single deoxyinosine to amplify Fragment 1 and Fragment 2 respectively are shown in bold type. Deoxyinosine (I) base is shown as an underlined base.

Fragment 1

(SEQ ID NO: 18)

5′ . . . GAGGTCACCGCCATCGCAGATCGCTAG-3′

(SEQ ID NO: 19)

3′ . . . CTCCAGTGGCGGTAGCGTCTAGCGATC-5′

(SEQ ID NO: 20)

3′-CTCCAGTGGCGITAGCGTCTAGCGATC-5′ (R1)
Fragment 2

(SEQ ID NO: 21)

(F2) 5′-CATCGCAGATCGCTAICAATATCAGGAGATTTTG-3′

(SEQ ID NO: 22)

5′-CTTCGCAGATCGCTAGCAATATCAGGAGATTTTG . . . 3′

(SEQ ID NO: 23)

3′-GTAGCGTCTAGCGATCGTTATAGTCCTCTAAAAC . . . 5′

Step 3: This step involves PCR amplification of the synthetic 10 Kb adjoining overlapping DNA fragments using the designed primers and a thermophilic DNA polymerase, preferably one that does not add an “A” nucleotide to the 3′ end of the amplified PCR product. Only the overlapping sequences of the two fragments are shown.

Fragment 1

(SEQ ID NO: 18)

5′ . . . GAGGTCACCGCCATCGCAGATCGCTAG-3′

(SEQ ID NO: 20)

3′ . . . CTCCAGTGGCGITAGCGTCTAGCGATC-5′
Fragment 2

(SEQ ID NO: 21)

5′-CATCGCAGATCGCTAICAATATCAGGAGATTTTG . . . 3′

(SEQ ID NO: 23)

3′-GTAGCGTCTAGCGATCGTTATAGTCCTCTAAAAC . . . 5′

Step 4: This step involves selective removal of deoxyinosine to form an abasic site, followed by phosphodiester backbone cleavage of the PCR amplified product by chemical treatment to generate long compatible ends between adjoining overlapping 10-Kb DNA fragments.

Fragment 1

(SEQ ID NO: 18)

5′ . . . GAGGTCACCGCCATCGCAGATCGCTAG-3′

(SEQ ID NO: 24)

3′ . . . CTCCAGTGGCG-5′
Fragment 2

(SEQ ID NO: 25)

5′-CAATATCAGGAGATTTTG . . . 3′

(SEQ ID NO: 23)

3′-GTAGCGTCTAGCGATCGTTATAGTCCTCTAAAAC . . . 5′

Step 5: Ligation of the 10-Kb fragments with compatible ends using T4 DNA ligase (or thermostable ligase) results in the formation of a 20-Kb DNA segment.

(SEQ ID NO: 16)

5′ . . . GAGGTCACCGCCATCGCAGATCGCTAGCAATATCAGGAGAT
TTTG . . . 3′

(SEQ ID NO: 17)

3′ . . . CTCCAGTGGCGGTAGCGTCTAGCGATCGTTATAGTCCTCTA
AAAC . . . 5′

### Example 3

### Assembly of a 30 Kb Segment from Three Overlapping 10 Kb Fragments

In the Example herein, a novel chemical process and composition to assemble a series of three (or more) overlapping adjoining 10-Kb DNA fragments into a DNA metasegment is described.

Step 1: Between the adjoining segments, the sequence overlap is chosen such that the plus strands terminate with a G at the 3′ end, which is separated by about 5-15 nucleotides from a C nucleotide upstream (G and C bases are in boldface). The DNA sequence at the junction after assembly of three 10-Kb fragments is shown

(SEQ ID NO: 26)

5′ . . . NNN . . . ATCGCAGATCGCTAGCAAT . . . NNNNN
NNNNNNNNN . . . AAGACACGGCCGGGT . . . NNN . . . 3′

(SEQ ID NO: 27)

3′ . . . NNN . . . TAGCGTCTAGCGATCGTTA . . . NNNNN
NNNNNNNNN . . . TTCTGTGCCGGCCCA . . . NNN . . . 5′

Step 2: Overlapping ends of the three chemically synthesized 10-Kb fragments or PCR fragments amplified from naturally occurring DNA that are to be assembled into a 30-Kb DNA segment are provided. Reverse (R1, R2) and forward (F2, F3) PCR primer designs with a single deoxyinosine to amplify Fragment 1, Fragment 2 and Fragment 3, respectively, are shown in bold type. Deoxyinosine base is shown as an underlined base.

Fragment 1

(SEQ ID NO: 28)

5′ . . . NNN . . . ATCGCAGATCGCTAGCAAT-3′

(SEQ ID NO: 29)

3′ . . . NNN . . . TAGCGTCTAGCGATCGAAT-5′

(SEQ ID NO: 30)

3′ . . . NNN . . . TAICGTCTAGCGATC-5′ (R1)
Fragment 2

(SEQ ID NO: 31)

(F2) 5′-CGCAGATCGCTAICAAT-3′

(SEQ ID NO: 32)

5′-ATCGCAGATCGCTAGCAAT . . . NNNNNNNNNNNNNN . . .
AAGACACGGCCGGGT . . . 3′

(SEQ ID NO: 33)

3′-TAGCGTCTAGCGATCGTTA . . . NNNNNNNNNNNNNN . . .
TTCTGTGCCGGCCCA . . . 5′

(SEQ ID NO: 34)

3′-TTCTITGCCGGCCC-5′ (R2)
Fragment 3

(SEQ ID NO: 35)

(F3) 5′-CACGGCCGGIT . . . NNN . . . 3′

(SEQ ID NO: 36)

5′-AAGACACGGCCGGGT . . . NNN . . . 3′

(SEQ ID NO: 37)

3′-TTCTGTGCCGGCCCA . . . NNN . . . 5′

Step 3: PCR amplification of the three adjoining overlapping synthetic 10 Kb DNA fragments using the designed primers and a thermostable DNA polymerase, preferably one that does not add an “A” nucleotide to the 3′ end of the amplified PCR product is performed. Only the overlapping sequences of the three DNA fragments are shown.

Fragment 1

(SEQ ID NO: 38)

5′ . . . NNN . . . CGCAGATCGCTAG-3′

(SEQ ID NO: 39)

3′ . . . NNN . . . ICGTCTAGCGATC-5′
Fragment 2

(SEQ ID NO: 40)

5′-CGCAGATCGCTAICAAT . . . NNNNNNNNNNNNNN . . .
AAGACACGGCCGGG-3′

(SEQ ID NO: 41)

3′-GCGTCTAGCGATCGTTA . . . NNNNNNNNNNNNNN . . .
TTCTITGCCGGCCC-5′
Fragment 3

(SEQ ID NO: 35)

5′-CACGGCCGGIT . . . NNN . . . 3′

(SEQ ID NO: 42)

3′-GTGCCGGCCCA . . . NNN . . . 5′

Step 4: Selective removal of deoxyinosine to form an abasic site, followed by phosphodiester backbone cleavage of the PCR amplified product by chemical treatment to generate long compatible single strand ends between adjoining overlapping 10-Kb DNA fragments is shown below.

Fragment 1

(SEQ ID NO: 43)

5′ . . . NNN . . . ATCGCAGATCGCTAG-3′
3′ . . . NNN . . . TA-5′
Fragment 2

(SEQ ID NO: 44)

5′-CAAT . . . NNNNNNNNNNNNNN . . . AAGACACGGCCGGG-
3′

(SEQ ID NO: 45)

3′-GCGTCTAGCGATCGTTA . . . NNNNNNNNNNNNNN . . . TT
CT-5′
Fragment 3
5′-T . . . NNN . . . 3′

(SEQ ID NO: 42)

3′-GTGCCGGCCCA . . . NNN . . . 5′

Step 5: Ligation of the three 10-Kb fragments with compatible single strand ends using T4 DNA ligase (or thermostable ligase) to form a 30-Kb DNA metasegment is shown below

(SEQ ID NO: 26)

5′ . . . NNN . . . ATCGCAGATCGCTAGCAAT . . . NNNNN
NNNNNNNNN . . . AAGACACGGCCGGGT . . . NNN . . . 3′

(SEQ ID NO: 27)

3′ . . . NNN . . . TAGCGTCTAGCGATCGTTA . . . NNNNN
NNNNNNNNN . . . TTCTGTGCCGGCCCA . . . NNN . . . 5′

### Example 4

### Assembly of a 20 Kb Segment from Two Overlapping 10 Kb Fragments with U Containing Primers

In the example described herein, a novel enzymatic process to assemble two overlapping 10 Kb fragments to form a 20 Kb segment is described. The uracil excision/abasic phosphodiester backbone cleavage from the PCR-amplified product is achieved enzymatically by using UDG and Endo VIII.

Step 1: Between the adjoining segments, the sequence overlap is chosen such that the plus strands terminate with T at the 3′ end, which is separated by about 5-15 nucleotides from A nucleotide upstream (The A and T are in boldface).

(SEQ ID NO: 16)

5′ . . . GAGGTCACCGCCATCGCAGATCGCTAGCAATATCAGGAGAT
TTTG . . . 3′

(SEQ ID NO: 17)

3′ . . . CTCCAGTGGCGGTAGCGTCTAGCGATCGTTATAGTCCTCTA
AAAC . . . 5′

Step 2: Overlapping ends of the two chemically synthesized 10-Kb fragments or PCR fragments amplified from naturally occurring DNA that are to be assembled into a 20-Kb fragment are provided. Reverse (R1) and forward (F2) PCR primer designs with a single uracil to amplify Fragment 1 and Fragment 2 respectively are shown in bold type. Uracil (U) is shown as an underlined base.

Fragment 1

(SEQ ID NO: 46)

5′ . . . GAGGTCACCGCCATCGCAGATCGCTAGCAAT-3′

(SEQ ID NO: 47)

3′ . . . CTCCAGTGGCGGTAGCGTCTAGCGATCGTTA-5′

(SEQ ID NO: 48)

3′-CTCCAGTGGCGGUAGCGTCTAGCGATCGTTA-5′ (R1)
Fragment 2

(SEQ ID NO: 49)

(F1) 5′-ATCGCAGATCGCTAGCAAUATCAGGAGATTTTG-3′

(SEQ ID NO: 50)

5′-ATCGCAGATCGCTAGCAATATCAGGAGATTTTG . . . 3′

(SEQ ID NO: 51)

3′-TAGCGTCTAGCGATCGTTATAGTCCTCTAAAAC . . . 5′

Step 3: PCR amplification using the designed PCR primers and a thermophilic DNA polymerase, preferably one that does not add an “A” nucleotide to the 3′ end of the amplified PCR product occurs in this step.

Fragment 1

(SEQ ID NO: 46)

5′ . . . GAGGTCACCGCCATCGCAGATCGCTAGCAAT-3′

(SEQ ID NO: 48)

3′ . . . CTCCAGTGGCGGUAGCGTCTAGCGATCGTTA-5′
Fragment 2

(SEQ ID NO: 49)

5′-ATCGCAGATCGCTAGCAAUATCAGGAGATTTTG . . . 3′

(SEQ ID NO: 51)

3′-TAGCGTCTAGCGATCGTTATAGTCCTCTAAAAC . . . 5′

Step 4: Uracil excision/abasic phosphodiester backbone cleavage of the PCR amplified 10-Kb fragments using UDG and Endo VIII to generate unique 3′ and 5′ compatible single strand ends between adjoining tandem DNA segments is undertaken. The USER kit from New England Biolabs is available for performing this step.

Fragment 1

(SEQ ID NO: 46)

5′ . . . GAGGTCACCGCCATCGCAGATCGCTAGCAAT-3′

(SEQ ID NO: 52)

3′ . . . CTCCAGTGGCGG-5′
Fragment 2

(SEQ ID NO: 53)

5′-ATCAGGAGATTTTG . . . 3′

(SEQ ID NO: 51)

3′-TAGCGTCTAGCGATCGTTATAGTCCTCTAAAAC . . . 5′

Step 5: The 10-Kb fragments in this step are ligated with compatible ends using T4 DNA ligase (or thermostable ligase) to form a 20-Kb DNA segment is undertaken.

(SEQ ID NO: 16)

5′ . . . GAGGTCACCGCCATCGCAGATCGCTAGCAATATCAGGAGAT
TTTG . . . 3′

(SEQ ID NO: 17)

3′ . . . CTCCAGTGGCGGTAGCGTCTAGCGATCGTTATAGTCCTCTA
AAAC . . .5′

### Example 5

### Assembly of a 30 Kb Segment from Three Overlapping 10 Kb Fragments with U Containing Primers

In the example described herein, a novel enzymatic process to assemble three overlapping 10 Kb fragments to form a 30 Kb segment. The uracil excision/abasic phosphodiester backbone cleavage from the PCR-amplified product is achieved enzymatically by using UDG and Endo VIII.

Step 1: The junction sequences of the 30-Kb DNA segment that overlap the 3′ and 5′ adjoining ends of three synthetic 10-Kb fragments is provided. Between the adjoining segments, the sequence overlap is chosen such that the plus strands terminate with T at the 3′ end, which is separated by about 5-15 nucleotides from A nucleotide upstream (A and T are in boldface).

(SEQ ID NO: 26)

5′ . . . NNN . . . ATCGCAGATCGCTAGCAAT . . . NNNNN
NNNNNNNNN . . . AAGACACGGCCGGGT . . . NNN . . . 3′

(SEQ ID NO: 27)

3′ . . . NNN . . . TAGCGTCTAGCGATCGTTA . . . NNNNN
NNNNNNNNN . . . TTCTGTGCCGGCCCA . . . NNN . . . 5′

Step 2: Overlapping ends of the three chemically synthesized 10-Kb fragments or PCR fragments amplified from naturally occurring DNA that are to be assembled into a 30-Kb DNA segment are provided. Reverse (R1, R2) and forward (F2, F3) PCR primer designs with a single uracil to amplify Fragment 1, Fragment 2 and Fragment 3, respectively, are shown in bold type. Uracil (U) is shown as an underlined base.

Fragment 1

(SEQ ID NO: 28)

5′ . . . NNN . . . ATCGCAGATCGCTAGCAAT-3′

(SEQ ID NO: 29)

3′ . . . NNN . . . TAGCGTCTAGCGATCGAAT-5′

(SEQ ID NO: 54)

3′- . . . UAGCGTCTAGCGATCGAAb T-5′ (R1)
Fragment 2

(SEQ ID NO: 55)

(F2)5′-ATCGCAGATCGCTAGCAAU . . . -3′

(SEQ ID NO: 32)

5′-ATCGCAGATCGCTAGCAAT . . . NNNNNNNNNNNNNN . . .
AAGACACGGCCGGGT . . . 3′

(SEQ ID NO: 33)

3′-TAGCGTCTAGCGATCGTTA . . . NNNNNNNNNNNNNN . . .
TTCTGTGCCGGCCCA . . . 5′

(SEQ ID NO: 56)

3′- . . . UTCTGTGCCGGCCCA-5′ (R2)
Fragment 3

(SEQ ID NO: 57)

(F3) 5′-AAGACACGGCCGGGU . . . 3′

(SEQ ID NO: 36)

5′-AAGACACGGCCGGGT . . . NNN . . . 3′

(SEQ ID NO: 37)

3′-TTCTGTGCCGGCCCA . . . NNN . . . 5′

Step 3: PCR amplification of the three adjoining overlapping synthetic 10 Kb DNA fragments using the designed primers and a thermophilic DNA polymerase, preferably one that does not add an “A” nucleotide to the 3′ end of the amplified PCR product is undertaken. Only the overlapping sequences of the three DNA fragments are shown.

Fragment 1

(SEQ ID NO: 28)

5′ . . . NNN . . . ATCGCAGATCGCTAGCAAT-3′

(SEQ ID NO: 58)

3′ . . . NNN . . . UAGCGTCTAGCGATCGTTA-5′
Fragment 2

(SEQ ID NO: 59)

5′-ATCGCAGATCGCTAGCAAU . . . NNNNNNNNNNNNNN . . .
AAGACACGGCCGGGT-3′

(SEQ ID NO: 60)

3′-TAGCGTCTAGCGATCGTTA . . . NNNNNNNNNNNNNN . . .
UTCTGTGCCGGCCCA-5′

| | |
|---|---|
| Fragment 3 | |
| 3′ | |
| (SEQ ID NO: 61) | 5′-AAGACACGGCCGGGU . . . NNN . . . |
| 5′ | |
| (SEQ ID NO: 37) | 3′-TTCTGTGCCGGCCCA . . . NNN . . . |

Step 4: Uracil excision/abasic phosphodiester backbone cleavage of the PCR amplified 10-Kb fragments using UDG and Endo VIII is performed. The USER kit from New England Biolabs is available for performing this step.

Fragment 1

(SEQ ID NO: 28)

5′ . . . NNN . . . ATCGCAGATCGCTAGCAAT-3′
3′ . . . NNN . . . -5′
Fragment 2

(SEQ ID NO: 62)

5′- . . . NNNNNNNNNNNNNN . . . AAGACACGGCCGGGT-3′

(SEQ ID NO: 63)

3′-TAGCGTCTAGCGATCGTTA . . . NNNNNNNNNNNNN
N . . . -5′
Fragment 3
5′- . . . NNN . . . 3′

(SEQ ID NO: 37)

3′-TTCTGTGCCGGCCCA . . . NNN . . . 5′

Step 5: Ligation of the 10-Kb fragments with compatible ends using T4 DNA ligase or thermostable ligase to form a 30-Kb DNA segment is performed.

(SEQ ID NO: 26)

5′ . . . NNN . . . ATCGCAGATCGCTAGCAAT . . . NNNNN
NNNNNNNNN . . . AAGACACGGCCGGGT . . . NNN . . . 3′

(SEQ ID NO: 27)

3′ . . . NNN . . . TAGCGTCTAGCGATCGTTA . . . NNNNN
NNNNNNNNN . . . TTCTGTGCCGGCCCA . . . NNN . . . 5′

### Example 6

### Creation of Metasegments Using Uracil Containing Primers

This example describes obtaining products from the ligation reaction of a series of PCR amplified 0.5-10 Kb overlapping DNA fragments using forward and reverse primers containing uracil, then treating with USER enzymes to produce long unique 3′ and 5′ single strand ends between adjoining fragments.

### Example 6A

### Starting Material: Three Synthetic 10 KB Fragments

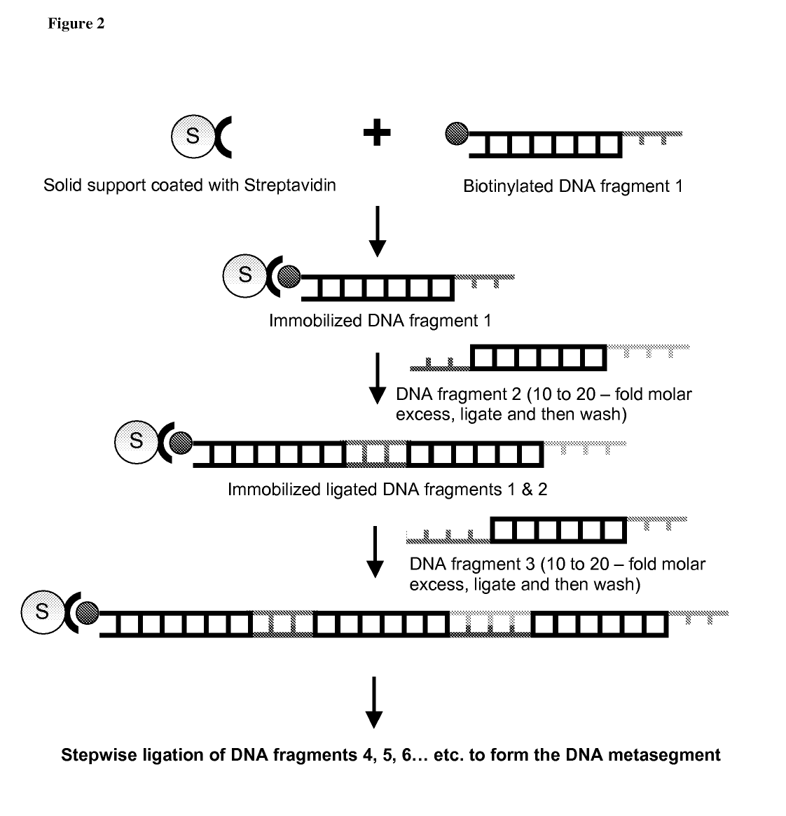

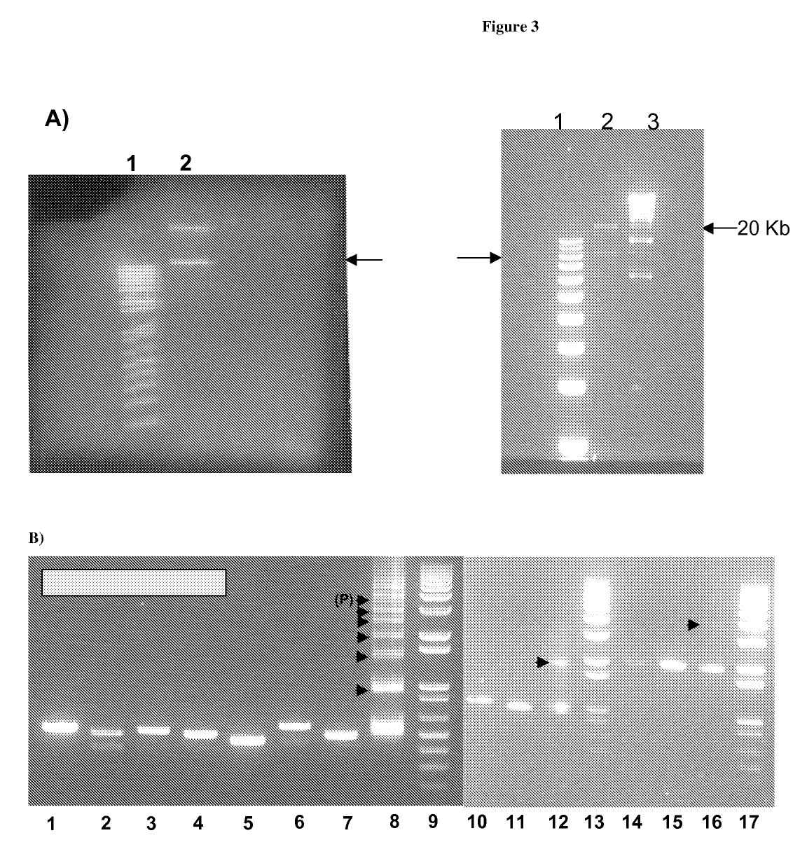

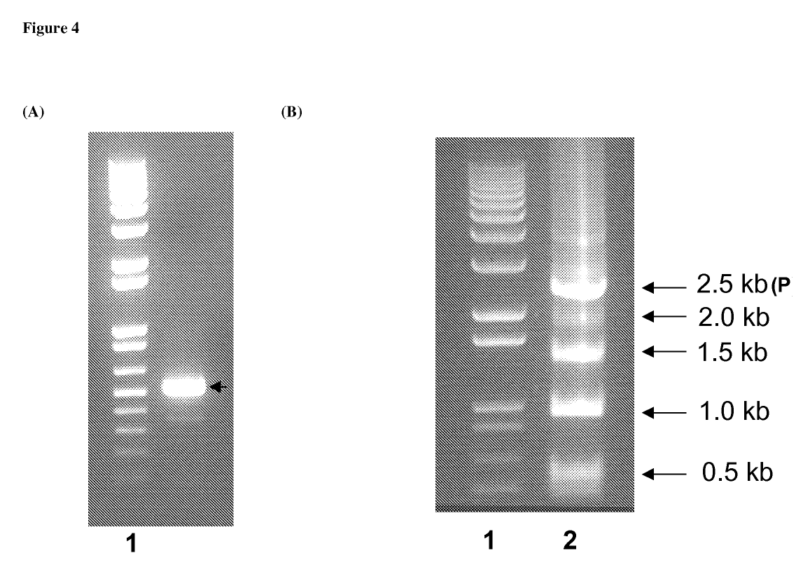

Three synthetic 10-Kb DNA fragments from yeast genome are amplified using Phusion High Fidelity Hot Start DNA polymerase and the designed PCR primers (F1: 5′-GGAGACAUAAATCTTT TGCTCTCTCT TCCTGC-3′ (SEQ ID NO:64) and R1: 5′-GATATTGCTAGCGAUCTGCGATGGCGGTGACCT-3′ (SEQ ID NO:65) for Fragment 1; F2: 5′-ATCGCTAGC AATAUCAGGAGATTTTGATTTTTTG-3′ (SEQ ID NO:66) and R2: 5′-CTCACCCGGC CGTGUCT TCACTA AACTCCTA GC-3′ (SEQ ID NO:67) for Fragment 2; and F3: 5′-AAGACACGGCCGGGUGAGAATTGGTTTTCTTTC-3′ (SEQ ID NO:68) and R3: 5′-GGGAAAGUTTAATTTCTTGAAATTTTCCAGAT-3′ (SEQ ID NO: 69) for Fragment 3). The PCR primer designs F1 and R3 have sequences incorporated in them at the 5′ end to enable cloning of the assembled DNA metasegment using the USER Friendly Cloning Kit from New England Biolabs. The agarose gel profiles of amplified PCR products 1 and 2 are shown in FIG. 3A (Lanes: 1, 1 kB marker and 2, PCR product from amplification of the fragment 1 from the yeast genome). The PCR-amplified products from Fragment 1 and Fragment 2 are treated with USER enzyme purchased from New England Biolabs to generate unique long single-strand extensions between adjoining fragments and then ligated using T4 DNA ligase, the agarose profile of which is shown in B (Lanes: 1, 1 Kb ladder; 2, ligation mixture; and 3, High MW markers. The expected product is indicated by the arrow. All three fragments, when ligated using T4 DNA ligase, produces a 30-Kb DNA metasegment, which will be then used as a substrate for homologous recombination in yeast. The 30-Kb DNA metasegment contains a selectable marker and is introduced into the yeast cell using standard molecular biology techniques.

### Example 6B

### Starting Material: Seven Synthetic 10 KB Fragments

The production of products (P) from ligation of seven overlapping ˜0.5-0.6 Kb (lanes 1-8); two 1 Kb (lanes 10-12); and two 2 Kb (lanes 14-16) fragments, respectively, are shown (see Table 1 for the corresponding forward and reverse primers used in the PCR amplification) in FIG. 3B. The ligation reaction in solution, yields a mixture of products, which are shown by arrows. Lanes 8, 13, and 17 show 1 Kb plus ladder.

### Example 7

### PCR Amplification Using DNA Primers Containing One or More RNA Nucleotides (I)

Four overlapping DNA fragments (˜0.5-0.6 Kb in size) are amplified using corresponding forward and reverse RNA/DNA primers and a thermostable DNA polymerase that has no terminal transferase activity (for example Pfu TurboCx). The PCR reaction condition is as follows:

| | |
|---|---|
| Template DNA (10-20 ng) = | 1 μl |
| Forward DNA primer containing RNA residues = | 5 μl |
| Reverse DNA primer containing RNA residues = | 5 μl |
| 10x buffer = | 10 μl |
| dNTPs (10 mM) = | 2 μl |
| Water = | 65 μl |
| Pfu Turbo Cx = | 2 μl |
| | 100 μl |

The amplification comprises 35 cycles with a denaturing step at 94° C. for 0.5 min, an annealing step at 55° C. for 0.5 min, followed by a polymerase extension step at 72° C. for 2 min. In an initial step before cyclic amplification, the DNA is denatured at 94° C. for 2 min. and the extension step after the final cycle occurs at 72° C. for 10 min.

To the above PCR amplified DNA, 10 μl of 3M sodium acetate is added and then extracted with 1× phenol-chloroform, 2× with chloroform and then precipitated with 2.5 volumes of ethanol. The solution is incubated at −80° C. 1-2 hours. The precipitate is spun down, washed with 70% ethanol, and air dried.

The precipitate is re-suspended in 36 μl DEPC treated water, 10 μl 5× Monsterscript Reverse Transcriptase buffer, 2 μl dNTPs (5 mM) and 2 μl (100 units) MonsterScript Reverse Transcriptase from Epicentre Biotechnologies (or Thermo-X Reverse Transcriptase from Invitrogen, Inc.). The reverse transcription through the RNA nucleotides of the primer segments is carried out at 60° C. for 60 min. Alternatively, the PCR amplification of the overlapping DNA segments that are to be assembled using a thermophilic DNA polymerase containing inherent reverse transcriptase activity in a one-step reaction (Shandilya et al. 2004; U.S. Pat. No. 6,030,814).

The PCR products are then subjected to a QIAGEN purification step. The purified PCR product, that is amplified using Pfu Turbo Cx and followed by treatment with MonsterScript reverse transcriptase and dNTPs, is then subjected to the RNaseH treatment to cleave at the ribonucleotides to generate PCR products with unique 5′ and 3′ overhangs. The overlapping DNA fragments are then ligated using T4 DNA ligase (or a thermostable ligase) as discussed above.

### Example 8

### PCR Amplification Using DNA Primers Containing an RNA Segment

In the example described herein, the assembly of a 30 Kb metafragment from three DNA fragments using DNA/RNA primers is described.

Step 1: The chosen sequence overlap between adjoining segments is about 20-30 bp, for which the DNA primers containing one or more ribonucleotide(s) are designed. An overlap is chosen such that the plus strands terminate with a G at the 3′ end, which is separated by about 5-15 nucleotides from a C nucleotide upstream (G and C bases are in boldface). DNA primers containing one or more ribonucleotides are then designed to the overlapping sequence of about 5-15 nucleotides. The DNA primers containing one or more ribonucleotides could be designed for any sequence of the overlapping segment and need not be defined by defined by G/C nucleotide that is they don't have to be specified either by G&C or A&T nucleotides as in Examples 2-3 and 6.

(SEQ ID NO: 26)

5′ . . . NNN . . . ATCGCAGATCGCTAGCAAT . . . NNNNN
NNNNNNNNN . . . AAGACACGGCCGGGT . . . NNN . . . 3′

(SEQ ID NO: 27)

3′ . . . NNN . . . TAGCGTCTAGCGATCGTTA . . . NNNNN
NNNNNNNNN . . . TTCTGTGCCGGCCCA . . . NNN . . . 5′

Step 2: Overlapping ends of the three chemically synthesized 10-Kb fragments or PCR fragments amplified from naturally occurring DNA that are to be assembled into a DNA metasegment are provided. Reverse (R1, R2) and forward (F2, F3) RNA-DNA PCR primer designs are used to amplify Fragment 1, Fragment 2 and Fragment 3, respectively, are shown in bold type. The ribonucleotide segment of the PCR primers are in boldface. Alternatively, just a single underlined G nucleotide could be incorporated as a ribonucleotide for cleavage by RNaseH while others in the primers are deoxyribonucletides with T substituting for U.

Fragment 1

(SEQ ID NO: 28)

5′ . . . NNN . . . ATCGCAGATCGCTAGCAAT-3′

(SEQ ID NO: 29)

3′ . . . NNN . . . TAGCGTCTAGCGATCGAAT-5′

(SEQ ID NO: 70)

3′ . . . NNN . . . TAGCGUCUAGCGAUC-5′ (R1)
Fragment 2

(SEQ ID NO: 71)

(F2) 5′-CGCAGAUCGCUAGCAAT-3′

(SEQ ID NO: 32)

5′-ATCGCAGATCGCTAGCAAT . . . NNNNNNNNNNNNNN . . .
AAGACACGGCCGGGT . . . 3′

(SEQ ID NO: 33)

3′-TAGCGTCTAGCGATCGTTA . . . NNNNNNNNNNNNNN . . .
TTCTGTGCCGGCCCA . . . 5′

(SEQ ID NO: 72)

3′-TTCTGUGCCGGCCC-5′ (R2)
Fragment 3

(SEQ ID NO: 70)

(F3) 5′-CACGGCCGGGT . . . NNN . . . 3′

(SEQ ID NO: 36)

5′-AAGACACGGCCGGGT . . . NNN . . . 3′

(SEQ ID NO: 37)

3′-TTCTGTGCCGGCCCA . . . NNN . . . 5′

Step 3: PCR amplification of the three adjoining overlapping synthetic 10 Kb DNA fragments using the designed ribonucleotide primers and a thermostable DNA polymerase, preferably one that does not add an “A” nucleotide to the 3′ end of the amplified PCR product, followed by extension using a thermostable reverse transcriptase is performed. Only the overlapping sequences of the three DNA fragments are shown.

Fragment 1

(SEQ ID NO: 38)

5′ . . . NNN . . . CGCAGATCGCTAG-3′

(SEQ ID NO: 71)

3′ . . . NNN . . . GCGUCUAGCGAUC-5′
Fragment 2

(SEQ ID NO: 72)

5′-CGCAGAUCGCUAGCAAT . . . NNNNNNNNNNNNNN . . .
AAGACACGGCCGGG-3′

(SEQ ID NO: 73)

3′-GCGTCTAGCGATCGTTA . . . NNNNNNNNNNNNNN . . .
TTCTGUGCCGGCCC-5′
Fragment 3

(SEQ ID NO: 70)

5′-CACGGCCGGGT . . . NNN . . . 3′

(SEQ ID NO: 42

3′-GTGCCGGCCCA . . . NNN . . . 5′

Step 4: Selective removal of ribonucleotides by RNases (RNaseH) to generate long compatible single strand ends between adjoining overlapping 0.5 to 10-Kb DNA fragments is performed.

Fragment 1

(SEQ ID NO: 43)

5′ . . . NNN . . . ATCGCAGATCGCTAG-3′
3′ . . . NNN . . . TA-5′
Fragment 2

(SEQ ID NO: 44)

5′-CAAT . . . NNNNNNNNNNNNNN . . . AAGACACGGCCGGG-
3′

(SEQ ID NO: 45)

3′-GCGTCTAGCGATCGTTA . . . NNNNNNNNNNNNNN . . . TT
CT-5′
Fragment 3

(SEQ ID NO: 42)

5′-T . . . NNN . . . 3′
3′-GTGCCGGCCCA . . . NNN . . . 5′

Step 5: Ligation of the three 0.5 to 10-Kb fragments with compatible single strand ends using T4 DNA ligase or a thermostable ligase to form the DNA metasegment.

(SEQ ID NO: 26)

5′ . . . NNN . . . ATCGCAGATCGCTAGCAAT . . . NNNNN
NNNNNNNNN . . . AAGACACGGCCGGGT . . . NNN . . . 3′

(SEQ ID NO: 27)

3′ . . . NNN . . . TAGCGTCTAGCGATCGTTA . . . NNNNN
NNNNNNNNN . . . TTCTGTGCCGGCCCA . . . NNN . . . 5′

### Example 9

### Automation of DMSAP

A schematic representation is shown in FIG. 2 and is described below.

### Step 1: PCR Amplification of the 5′ Biotinylated DNA Segment 1

PCR amplification of the fragment that is to be attached solid support (Dynabeads Streptavidin beads from Invitrogen) is amplified using the corresponding 5′ biotinylated forward primer, the reverse primer (containing uracil) and a thermostable DNA polymerase that has no terminal transferase activity (like Pfu Turbo Cx). The PCR reaction condition is as follows:

| | |
|---|---|
| Template DNA (10-20 ng) = | 1 μl |
| 5′ biotinylated forward primer (2 μM) = | 5 μl |
| Reverse primer (containing uracil) (2 μM) = | 5 μl |
| 10x buffer = | 10 μl |
| dNTPs (10 mM) = | 2 μl |
| Water = | 65 μl |
| Pfu Turbo Cx = | 2 μl |
| | 100 μl |

The amplification comprises 35 cycles with a denaturing step at 94° C. for 0.5 min, an annealing step at 55° C. for 0.5 min, followed by a polymerase extension step at 72° C. for 2 min. In an initial step before cyclic amplification the DNA is denatured at 94° C. for 2 min and in a final step after cyclic amplification, the polymerase extension was carried out at 72° C. for 10 min. The PCR products were then subjected to QIAtip purification step.

Step 2: Treatment of PCR Amplified 5′ Biotinylated Dna Fragment 1 with USER Enzymes

The PCR-amplified 5′ biotinylated DNA is then treated with USER enzymes to generate a unique 3′ single strand end. To the PCR mix, 10 μl of 3M sodium acetate solution is added and extracted once, with an equal volume of phenol-chloroform, twice with chloroform and then precipitate with 2.5 volumes of ethanol. The solution is then incubated at −80° C. for 1 hr. The resulting precipitate was then spun down using a microcentrifuge at 4° C., washed with 70% ethanol and then air dried for 20 minutes in the hood.

The precipitate is resuspended in 13 μl of water.

Add 2 μl of Taq 10×PCR buffer (from Applied Biosystems, Inc).
1 μl of 100 mM DTT (dithiothreitol)
4 μl of USER enzyme (from NEB)

The mixture is incubated at 37° C. overnight. Next day, 40 μl of water and 5 μl of 3M sodium acetate is added and phenol-chloroform extraction is conducted followed by ethanol precipitation as discussed above. Re-suspend the 5′ biotinylated DNA treated with USER enzymes in 20 μl of Binding Solution from Dynal kilobaseBINDER Kit.

### Step 3: Immobilization of 5′ Biotinylated DNA Fragment 1 to the Solid Support

The 5′ biotinylated DNA segment is then immobilized to Dynabeads Streptavidin (from Invitrogen) as described in the Dynal kilobaseBINDER Kit. Re-suspend the Dynabeads M-280 Streptavidin by vortexing the vial (purchased from Dynal, Invitrogen) to obtain a homogenous suspension. 5 μl (50 μg) is transferred to a 1.5 ml microcentrifuge tube. The tube is placed on the magnet for 2 min, and the supernatant is carefully pipetted off the supernatant. The tube is removed from the magnet add 20 μl of the Binding Solution is added. Beads are resuspended by pipetting. The tube is placed on the magnet and the supernatant is carefully removed. Beads are resuspended in 20 μl Binding Solution. The 5′ biotinylated DNA (10-20 picomoles) is added from Step 2 to resuspended beads and mixed carefully. The tube is incubated 3-6 hours at room temperature 20-25° C. on a roller to keep the beads in suspension. Then, the tube is placed on the magnet and the supernatant is removed as above. The Dynabeads/DNA complex is washed twice in 20 μl of Washing Solution and once in distilled water.

### Step 4: USER Reaction of the Overlapping DNA Fragment 2

The overlapping DNA fragment 2 (containing about 100-200 picomoles) that is to be sequentially ligated to the 5′ biotinylated DNA segment (immobilized on the beads) is amplified by PCR using the corresponding forward and reverse primers containing uracil and Pfu Turbo Cx in a 100 μl reaction. 10 μl of 3M sodium acetate is added to PCR mix and extract with an equal volume of phenol-chloroform, twice with chloroform and then precipitated with 2.5 volumes of ethanol and incubated at −80° C. for 1 hr. The resulting precipitate is then spun down using a microcentrifuge at 4° C., washed with 70% ethanol and then air dried for 20 minutes in the hood.

The precipitate is resuspended in 13 μl of water.

Add 2 μl of Taq 10×PCR buffer (from Applied Biosystems, Inc).
1 μl of 100 mM DTT (dithiothreitol)
4 μl of USER enzyme (from NEB)

The mixture is incubated at 37° C. overnight. Next day, 40 μl of water and 5 μl of 3M sodium acetate are added and phenol-chloroform extraction is conducted followed by ethanol precipitation as discussed above.

### Step 5: Annealing of DNA Fragment 2 to the Immobilized 5′ Biotinylated DNA Segment 2

The USER product (10-20 fold excess) from Step 2 is resuspended in 13 μl of water and 4 μl of 5× ligase buffer (from Invitrogen, Inc). The solution is heated at 60° C. for 10 minutes, and added to the 5′ biotinylated DNA segment that is immobilized on the Dynal Streptavidin beads. The mixture is allowed to cool down slowly to room temperature overnight.

Step 6: Stepwise Ligation of DNA Fragment 2 with Immobilized 5′ Biotinylated DNA Fragment 1

Next day, a the following is added to the ligation mix:
1 μl of 100 mM DTT
1 μl of 10 mM ATP
1 μl of T4 DNA ligase (from NEB)

The ligation mix is incubated at 16° C. overnight. The next day, the eppendorf tube is placed on the magnet for 2 min, and the supernatant is carefully pipetted off. The Dynabeads/DNA complex is washed twice in 20 μl of Washing Solution and once in distilled water. The excess unligated DNA segment 2 thus is washed away from the immobilized support containing the desired ligated product. The Dynabeads/DNA complex is now ready for the sequential addition of the next overlapping DNA fragment 3.

Step 7: Sequential or Stepwise Ligation of DNA Fragment 3 with Immobilized 5′ Biotinylated DNA Fragments 1 & 2

Steps 4 to 6 are repeated to sequentially ligate each of the subsequent overlapping DNA segments to the immobilized 5′ biotinylated DNA segments till the desired final DNA metasegment product (DMSAPP) is assembled on the solid support.

### Step 8: Release of the Immobilized DNA MetaSegment Assembly Process Product (DMSAPP) From the Solid Support.

The immobilized biotinylated DMSAPP can be released by incubating the mixture at 65° C. for 5 minutes or 2 min at 90° C. in 10 mM EDTA pH 8.2 with 95%, which will typically dissociate >90% of the immobilized biotinylated DNA.

It should be noted that the 5′ biotinylated DNA fragment 1 can easily be immobilized on streptavidin attached to solid or polymer support, which then is placed in a column and the sequential steps of adding each new fragment can be carried out. The excess unreacted reagents will then be washed away after each step. The DNA synthesizer from ABI and other companies could be readily modified for automated and parallel large scale synthesis of several DNA metasegments at a time using the DMSAP.

The DNA segments containing the unique 5′ and 3′ ends could be generated using the three different protocols described above.

This invention may be embodied in other forms or carried out in other ways without departing from the spirit or essential characteristics thereof. The present disclosure is therefore to be considered as in all aspects illustrate and not restrictive, and all changes which come within the meaning and range of equivalency are intended to be embraced therein.

Various references are cited throughout this specification, each of which is incorporated herein by reference in its entirety.

### REFERENCES

Bibikova M, Carroll D, Segal, D J, Trautman J K, Smith J, Kim Y G, Chandrasegaran S (2001). Stimulation of homologous recombination through targeted cleavage by chimeric nucleases. Mol Cell Biol 21: 289-297.
Bibikova M, Beumer K, Trautman J K, Carroll D (2003) Enhancing gene targeting with designed zinc finger nucleases. Science 300: 764.
Bitinaite J, Rubino M, Varma K H, Schildkraut I, Vaisvila R and Vaiskunaite (2007). USER™ friendly DNA engineering and cloning by uracil excision. Nucleic Acids Research 35: 1992-2002.
Chin, J. W. (2006) Modular approaches to expanding the functions of living matter. Nature Chemical Biology 2: 304-311.
Durai S, Mani M, Kandavelou K Porteus M, Chandrasegaran S (2005). Zinc finger nucleases: Custom designed molecular scissors for genome engineering of plant and mammalian cells. Nucleic Acids Research 33: 5978-5990.
Endy, D. (2005). Foundations for Engineering Biology. Nature 438: 449-453.
Geu-Flores F, Nour-Eldin H H, Nielsen M T and Halkier B A (2007) USER fusion: a rapid and efficient method for simultaneous fusion and cloning multiple PCR products. Nucleic Acids Research, 2007, e55, (doi:10.1093/nar/gkm 106).
Kandavelou K, Mani M, Durai S, Chandrasegaran S (2005). ‘Magic’ scissors for genome surgery. Nature Biotechnology 23: 686-687.
Kandavelou K, Mani M, Durai S, Chandrasegaran, S. (2004). Engineering and Applications of Chimeric Nucleases. In: Nucleic Acids and Molecular Biology, Vol. 14, Pingoud, A. M. (Ed.) Restriction endonucleases, pp. 413-434, Springer-Verlag, Berlin, Heidelberg, Germany.
Kumar S, Allen G C, Thompson W F (2006) Gene targeting in plants: fingers on the move. Trends in Plant Science 11: 159-161.
Kupfer P A, Leumann C J. (2007). The chemical stability of abasic RNA compared to abasic DNA. Nucleic Acids Research 35:58-68.
Lartigue C, Glass J I, Alperovich N, Pieper R, Parmar P P, Hutchison III C A, Smith H O, Venter J C (2007) Sciencexpress/www.sciencexpress.org/28 Jun. 2007/Page 1-6/10.1126/1144622.
Lasken R S, Schuster D M and Rashtchian A. (1996). Archaebacterial DNA polymerases tightly bind Uracil-containing DNA. Journal of Biological Chemistry 271: 17692-17696.
Maxam A M, Gilbert W (1980) Sequencing end-labeled DNA with base-specific chemical cleavages. Methods in Enzymology 65: 499-560.
Maxam A M, Gilbert W (1977) A new method for sequencing DNA. Proc. Natl. Acad. Sci., USA, 74: 560-564.
Menzella H G, Reid R, Carney J R, Chandran S S, Reisinger S J, Patel K G, Hopwood D A, Santi D V (2005) Combinatorial polyketide biosynthesis by de novo design and rearrangement of modular polyketide synthase genes. Nature Biotechnology 9: 1171-1176.
Porteus M H, Baltimore D (2003) Chimeric nucleases stimulate gene targeting in human cells. Science 300: 763.
Pósfai G, Plunkett III G, Fehér T, Frisch D, Keil G M, Umenhoffer, Kolisnychenko K V, Stahl B, Sharma S S, de Arruda M, Burland V, Harcum S W, Blattner F R (2006) Emergent properties of reduced genome Escherichia coli. Science 312: 1044-1046.
Reisinger S J, Patel K G, Santi D V (2006) Total synthesis of multi-kilobase DNA sequences from oligonucleotides. Nature Protocols 1: 2596-2603 (Published online 11 Jan. 2007, doi:10.1038/nprot.2006.426).
Shandilya H, Griffiths K, Flynn E K, Astatke M, Shih P-J, Lee J E, Gerard G F, Gibbs M D, Berquist P L (2004) hermophilic bacterial DNA polymerases with reverse transcriptase activity. Extremophiles 8: 243-251.
Smith H O, Hutchison III C A, PfannKoch C, Venter J C (2003) Generating a synthetic genome by whole genome assembly φX174 bacteriophage from synthetic oligonucleotides. Proc. Natl. Acad. Sci., USA, 100: 15440-15445.
Urnov F D, Miller J C, Lee Y L, Beausejour C M, Rock J M, Augustus S, Jamieson A C, Porteus M H, Gregory P D, Holmes M C. (2005) Highly efficient endogenous human gene correction using designed zinc-finger nucleases. Nature 435:646-651.
Wright D A, Townsend J A, Winfrey Jr. R J, Irwin P A, Rajagopal, J, Lonosky P M, Hall B D, Jondle, M D, Voytas, D F (2005) High-frequency of homologous recombination in plants mediated by zinc-finger nucleases. The Plant Journal 44:693-705.

What is claimed is:

1. A method of obtaining a DNA metasegment at least 10 Kb comprising:
(a) providing a plurality of adjoining DNA fragments wherein each fragment is at least 0.3 Kb in length and each adjoining fragment comprises an overlapping regions wherein said overlapping region comprises an overlap of at least 3 bp at the 5′ end and/or 3′ end between said adjoining fragments
(b) contacting the fragments provided in (a) with at least one forward primer and one reverse primer, wherein each primer is sufficiently complememtary to the overlap between said adjoining fragments to hybridize to at least one of said adjoining fragments, each primer comprises at least one removable base of at least 15 nucleotides in length;
(c) amplifying the DNA fragments of (a) using the primers of (b) to obtain amplified fragments, wherein the forward primer is used to amplify the plus strand of at least one of said fragments and the reverse primer is used to amplify the minus strand of at least one of said fragments;
(d) exposing amplified DNA of (c) to conditions that promote removal of the removable base and phosphodiester bond cleavage at the site of the removable base to produce DNA fragments at least 0.3 Kb in length with compatible single-stranded ends at least two nucleotides in length, wherein said compatible ends of said DNA fragments at least 3 nucleotides in length are sufficiently complementary to each other to hybridize to one another;
(e) ligating fragments with compatible single-stranded ends generated in (e) to obtain said DNA metasegments;
(f) optionally repeating steps (a)-(e) and
(g) isolating said metasegment.

2. The method according to claim 1, wherein said DNA fragments provided in (a) are obtained by chemical synthesis.

3. The method according to claim 1, wherein there is an overlap of 7-15 bp at the 5′ end and/or 3′ end between said adjoining fragments.

4. The method according to claim 1, wherein the overlapping region of the DNA fragments provided in (a) terminates with C at the 5′ end.

5. The method according to claim 1, wherein the overlapping region of the DNA fragments provided in (a) terminates with C at the 5′ end and further comprises G at least five nucleotides downstream from said G.

6. The method according to claim 1, the overlapping region of the DNA fragments provided in (a) terminates with A at the 5′ end.

7. The method according to claim 1, wherein the overlapping region of the DNA fragments provided in (a) terminates with A at the 5′ end and further comprises T at least five nucleotides downstream from said A.

8. The method according to claim 1, wherein at least one removable base in at least one primer provided in (b) is an acid labile base, photolabile base, or substrate for enzymatic removal.

9. The method according to claim 1, wherein at least one removable base in at least one primer provided in (b) is an acid labile base, wherein said acid labile base is deoxyinosine or N7-methyl deoxyguanine.

10. The method according to claim 1, wherein at least one removable base in at least one primer provided in (b) is a photolabile base, wherein said photolabile base.

11. The method according to claim 9, wherein at least one removable base in at least one primer provided in (b) is a substrate for enzymatic removal.

12. The method according to claim 1, wherein at least one removable base in at least one primer provided in (b) is uracil.

13. The method according to claim 12, wherein said uracil is a removed using a uracil excision reagent.

14. The method according to claim 12, wherein said uracil is removed and phosphodiester bond at said uracil is a uracil DNA glycosylase and DNA lyase Endo VIII.

15. The method according to claim 1, wherein at least one primer used in step (b) comprises at least one ribonucleotide or RNA fragment.

16. The method according to claim 1, wherein said ribonucleotide or ribonucleotide fragment is removed by RNase treatment.

17. The method according to claim 1, wherein said method is an automable method.

18. The method according to claim 1, wherein said DNA fragment is amplified in step (c) by polymerase chain reaction.

19. The method according to claim 1, wherein said DNA fragment is a thermostable polymerase.

20. The method according to claim 1, wherein at least one amplified sequence of step (c) is attached to a solid support.

21. The method according to claim 1 wherein the fragments in step (f) are ligated with a thermostable ligase.

22. A kit comprising at least one forward and reverse primer at least 12 nucleotides wherein the reverse primer is the reverse strand of the forward primer, and wherein each primer comprises a removable base, a DNA ligase, a DNA polymersase, nucleotide triphosphates and buffers.

23. The kit according to claim 22, wherein at least one primer is an RNA/DNA primer.

24. The kit according to claim 22, wherein the kit further comprises an RNase.

25. The kit according to claim 22, wherein the kit further comprises reverse transcriptase.

26. The kit according to claim 22, wherein the primers comprise C at the 5′ end.

27. The kit according to claim 22, wherein the primers comprise C at the 5′ end and I at least 5 nucleotides downstream from C.

28. The kit according to claim 22, wherein at least one primer is a 5′-biotinylated primer.

29. The kit according to claim 22, wherein the kit comprises streptavidin coated support.

30. An automatable method for obtaining a DNA metasegment at least 10 Kb comprising:
(a) providing a first DNA fragment at least 0.3 Kb in length;
(b) amplifying said first DNA fragment provided in (a), said amplification using a forward DNA primer containing a functional moiety to enable attachment of the amplified first 0.3 Kb fragment to a solid support at the 5′ end and a reverse primer, wherein said reverse primer amplifies the minus strand of the said first fragment from the 3′ end, wherein said reverse primer contains one or more removable bases and a DNA polymerase to produce a first amplification product;
(c) treating said amplification product of (b) to remove the removable base and cleave the phosphodiester bond at the site of the removable base to generate a first DNA fragment attached to the solid support having a unique 3′ single strand end, wherein said 3′ single strand end is at least 3 nucleotides in length;
(d) providing a second DNA fragment at least 0.3 Kb in length wherein at least three nucleotides at the 5′ end overlaps said first DNA fragment provided in (a) at the 3′ end;
(e) amplifying said second DNA fragment provided in (d), said amplification using a forward DNA primer and a reverse primer, wherein said forward primer amplifies the plus strand of said second DNA fragment and the reverse primer amplifies the minus strand of said second DNA fragment wherein said primers contain one or more removable bases and a DNA polymerase to obtain a second amplified product;
(f) treating said second amplified product of (e) to remove the removable base and cleave a phosphodiester bond at the site of said removable base to generate a second amplified fragment comprising (i) a reverse strand comprising a unique 5′ single strand end sufficiently complementary to the unique 3′ single stranded end of the first amplified DNA fragment attached to the solid support to hybridize to said 3′ single stranded end and (ii) a forward strand comprising a unique 3′ single strand end;
(g) ligating the amplified first DNA fragment to the amplified second DNA fragment comprising incubating at least a five fold excess of the amplified second DNA fragment with the amplified first DNA fragment in the presence of ligase to produce a first ligation product comprising a 3′ single strand end;
(h) providing a third DNA fragment at least 0.3 Kb in length, wherein at least three nucleotides at the 5′ end of said third DNA fragment overlaps with the 3′ single strand end of the ligation product of (g);
(i) amplifying said third DNA fragment provided in (h), said amplification using a forward DNA primer and a reverse primer, wherein said forward primer amplifies the plus strand of said third DNA fragment and said reverse primer amplifies the reverse strand of said third DNA fragment, wherein said primers contain one or more removable bases to obtain a third amplification product;
(j) treating said third amplification product to remove the removable base and cleave a phosphodiester bond at the site of said removable base to generate a third amplified fragment comprising (i) a reverse strand comprising a unique 5′ single strand end sufficiently complementary to the unique 3′ single stranded end of the first ligation product of (g) attached to the solid support to hybridize to said 3′ single stranded end and (ii) a forward strand comprising a unique 3′ single strand end;
(k) ligating the amplified third DNA fragment to the first ligation product of (g) comprising incubating at least a five fold and in particular, a 10 fold excess of the amplified third DNA fragment with the first ligation product in the presence of ligase to produce a second ligation product;
(l) optionally repeating steps (d)-(g); to obtain said DNA metasegment and
(m) removing the DNA metasegment obtained from the solid support.

31. The method according to claim 30, wherein said removable base is uracil.

32. The method according to claim 30, wherein said DNA polymerase in steps (b), (e) or (i) is a thermophilic DNA polymerase.

33. The method according to claim 30, wherein said DNA polymerase in steps (b), (e) or (i) is a thermophilic DNA polymerase without terminal transferase activity.

34. The method according to claim 30, wherein said DNA ligase in steps (g) or (k) is a thermophilic DNA ligase or a T4 DNA ligase.

35. The method according to claim 30, wherein the method further comprises a washing step after the ligation steps (g) and (k).

36. The method according to claim 30, wherein at least one primer used comprises at least one ribonucleotide or RNA fragment.
